(19) United States
(12) Patent Application Publication
(10) Pub. No.: US 2004/0121305 A1
(43) Pub. Date: Jun. 24, 2004

(54) Generation of efficacy, toxicity and disease signatures and methods of use thereof

(76) Inventors: Roger Charles Wiegand, Wayland, MA (US); Robert Michael McCarroll, Lexington, MA (US); Lily Y. T. Li, Lexington, MA (US); Dong Wei, Malden, MA (US); Merce Crosas, Lexington, MA (US); James Anthony Rogers, Hingham, MA (US); Alexander Frederic Rosenberg, Brookline, MA (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/323,493

(22) Filed: Dec. 18, 2002

(51) Int. Cl. C12Q 1/00

(52) U.S. Cl. 435/4

(57) ABSTRACT

The invention provides methods of generating small molecule efficacy profiles and signature, small molecule toxicity profiles and signatures and small molecule disease profiles and signatures. The invention also provides methods of determining the efficacy and/or toxicity of unknown agents and drugs in a subject and methods of diagnosing an unknown disease or disorder in a subject. The invention further provides methods of monitoring the progression or remission of a disease or disorder in a subject undergoing treatment and methods of measuring the effectiveness of treatment.

### FIELD OF THE INVENTION

This invention relates generally to methods for developing and assessing drug efficacy signatures, disease signatures and toxicity signatures using metabolomics.

### BACKGROUND OF THE INVENTION

Living organisms are autonomous chemical systems which include diverse sets of small molecules. Small molecules found in living systems include, for example, sugars, fatty acids, amino acids, nucleotides, and intermediates of metabolic and signaling pathways. Sugars are a primary source of chemical energy for cells. The cells break the sugars down through a series of oxidative reactions to small sugar derivatives and, ultimately, CO₂ and H₂O. Fatty acids used for both energy storage and as major components of cellular membranes. Amino acids are the building blocks of proteins. Nucleotides are involved in intracellular signaling, energy transfer, and as the monomers of the information macromolecules, RNA and DNA.

The cellular small molecules are, generally, composed of six elements (C, H, N, O, P, S). If water is excluded, carbon compounds comprise a large majority of the cellular small molecules. The cellular small molecules repeatedly use certain distinctive chemical groups, such as methyl (CH₃), carboxyl (COOH) and amino (NH₂) groups.

Generally, most cellular small molecules are synthesized from and broken down to the same basic compounds. Synthesis and metabolism occurs through sequences of controlled chemical reactions, catalyzed by enzymes. Most of the metabolic reactions of the cell occur in the cytoplasm, which contains many distinctive organelles. For example, the mitochondria are responsible for respiration and energy production. Mitochondria are the “power plants” of eukaryotic cells, harnessing energy contained by combining oxygen with metabolites to make ATP. Other organelles of the cell include the Golgi apparatus, a system of stacked, membrane bound, flattened sacs involved in modifying, sorting and packaging of macromolecules for secretion or for delivery to other organelles. The endoplasmic reticulum (ER) is a series of flattened sheets, sacks, and tubes of membrane extending throughout the cytoplasm of eukaryotic cells. The ER membrane is in structural continuity with the outer membrane of the nuclear envelope and specializes in the synthesis and transport of lipids and membrane proteins.

In recent years, scientists have attempted to study cells and living systems through the cataloging of the entire genome of an organism (e.g., genomics). Genomics is a powerful tool, useful for identifying and interrogating the entire inventory of genes of a living system. Recently, scientists have also attempted to identify and interrogate all the proteins present in the cell or organism through proteomics. However, most pharmaceutical companies that study genomics and proteomics realize that many of their anticipated products are not proteins nor genes but small molecules.

For example, once a novel gene or target is discovered by genomics, the investigators must first validate the target using expensive and time consuming procedures which are far removed from the actual disease state. Examples of typical validation procedures include expression profiling, generating knock-out mice or transgenic mice, in situ hybridization, etc. Once a target is validated, the investigators typically screen enormous random small molecule libraries to identify molecules which interact with the protein targets. The identified small molecules are typically optimized through chemical synthesis in order to obtain a marketable product.

Metabolomics eliminates much of the guesswork surrounding genomics. Small molecule signatures of cells and organelles can be used directly to identify drug candidates. Unlike genomics, small molecule profiling can either eliminate or accelerate the process of identifying genes and proteins associated with a disease state. For example, small molecule profiling allows one to investigate the very biochemical pathway (e.g., cellular metabolites) involved in the disease state by comparing small molecule signatures of cells, cellular compartments, or organelles with those of cells, cellular compartments, or organelles treated with toxins, chemical agents or other therapeutic agent (or derived from an organism treated with the agent or drug). Unlike genomics, small molecule profiling is not limited to disease states with a genetic component. Many disease states are not genetically determined and genomics offers little to those suffering or at risk of suffering from non-genetic linked disease states. Small molecule profiling of cells or organelles can be used to study both genetic and non-genetically linked disease states.

### SUMMARY OF THE INVENTION

In one embodiment, the present invention provides a method of generating a small molecule efficacy profile from a subject treated with an agent or drug, by isolating a biological source from the subject, and extracting and analyzing small molecules from the biological source, where analyzing the small molecules results in the generation of an efficacy profile.

In another embodiment, the present invention provides a method of generating a small molecule toxicity profile from a subject treated with a toxicant, by isolating a biological source from the subject, extracting and analyzing small molecules from the biological source, where analyzing the small molecules results in the generation of a toxicity profile.

In another embodiment, the present invention provides a method of generating a small molecule disease profile from a subject suffering from a disease or disorder, by isolating a biological source from a diseased subject, extracting and analyzing small molecules from the biological source from the diseased subject, where analyzing the small molecules results in the generation of a disease profile.

In another embodiment, the present invention provides a method of generating a small molecule efficacy signature from a subject, by obtaining a small molecule efficacy profile and comparing the small molecule efficacy profile to a small molecule efficacy profile, from an untreated subject, where that small molecule efficacy profile is generated from a similar biological source and comparing the small molecule efficacy profiles results in the generation of a small molecule efficacy signature.

In another embodiment, the present invention provides a method of generating a small molecule toxicity signature from a subject, by obtaining a small molecule toxicity profile and comparing the small molecule toxicity profile to a small molecule toxicity profile from an untreated subject, where that small molecule toxicity profile is generated from a similar biological source and comparing the small molecule toxicity profiles results in the generation of a small molecule toxicity signature.

In another embodiment, the present invention provides a method of generating a small molecule disease signature from a subject, by obtaining a small molecule disease profile and comparing the small molecule disease profile to a small molecule disease profile from a healthy subject, where that small molecule disease profile is generated from a similar biological source and comparing the small molecule disease profiles results in the generation of a small molecule disease signature.

In another embodiment, the present invention provides a method of determining the effectiveness of an agent or drug of in a subject, by obtaining an efficacy signature from a biological source from a subject following the subject's treatment with an unknown drug or agent and comparing that efficacy signature to an efficacy signature or a database of efficacy signatures generated from biological sources isolated from subjects treated with a range of known agents or drugs, thereby determining the effect of the unknown agent or drug on the subject and biological source.

In another embodiment, the present invention provides a method of determining the toxicity of an agent or drug in a subject, by obtaining a toxicity signature from a biological source from a subject following the subjects treatment with an agent or drug or unknown toxicity and comparing that toxicity signature to a toxicity signature or a database of toxicity signatures generated from biological sources isolated from subjects treated with a range of known toxicants, thereby determining the toxicity of the unknown agent or drug on the subject and biological source.

In another embodiment, the present invention provides a method of diagnosing a previously undiagnosed disease or disorder in a subject, by obtaining a disease signature from a diseased biological source from a subject with an unknown disease or disorder and comparing that disease signature to a disease signature or a database of disease signatures generated from biological sources obtained from subjects with a range of known diseases or disorders, thereby diagnosing the unknown disease or disorder in the subject.

In another embodiment, the invention provides a method of comparing small molecule efficacy, toxicity and disease profiles, by generating a data matrix including two or more analyte/sample values indicating small molecule abundance in the sample, log transforming the data matrix, normalizing the transforming data matrix which includes subtracting the median of all analyte/sample values from each analyte/sample value and performing variance analysis on the normalized data matrix, thereby generating small molecule efficacy, toxicity and disease profiles.

In another embodiment, the invention provides a method of monitoring the progression or remission of a disease or disorder in a subject undergoing treatment for that particular disease or disorder, by obtaining a small molecule disease profile from the subject at the onset of medical treatment for that particular disease or disorder, obtaining additional small molecule disease profiles at multiple times during the course of medical treatment for that particular disease or disorder and comparing those additional small molecule disease profiles with the small molecule disease profile obtained at the onset of medical treatment, thereby measuring the effectiveness or treatment and monitoring the progression or remission of the particular disease or disorder in the subject.

In another embodiment, the invention includes a biological source for generating a signature. The biological source can be an organ, tissue, cell, cellular compartment, organelle, blood or urine. The biological source can be from a subject. In a preferred embodiment, the subject is human. The subject can be a healthy reference subject or a subject that suffers from a disease or disorder. The disease or disorder can be non-insulin-dependent diabetes (NIDDM), rheumatoid arthritis or inflammation (RA/I), immunological disorder, metabolic disorder, cardiovascular disorder, neurological disorder, oncological disorder, or viral disorder.

In another embodiment, the invention includes an agent or drug. The agent or drug can be Chlorpropamide, Tolbuamide, Tolazamide, Acetohexamide, Glyburide, Glipizide, Glimepiride, Pioglitazone, Rosiglitazone, Metformin, Acarbose (Precose), Miglitol (Glycet), Repaglinide (Prandin), Aspirin, Acetaminophen, Ibuprofen, Indomethacin, Peroxicam, Tometin, Rofecoxib, Celecoxib, Valdecoxib, Methotrexate, or Dexamethasone.

In another embodiment, the invention includes a toxicant. The toxicant can be 2,2′,4,4′,5,5′-hexachlorobiphenyl (PCB-153), 2,3,7,8-tetrachlorodibenzo-p-dioxin (TCDD), 2-bromoethylamine (BEA), 3-methylcholanthrene, 4-aminophenol (PAP), acetaminophen, adriamycin, allyl alcohol, amiodarone, amphotericin B, Aroclor 1254, Aroclor 1260, arsenic, aspirin, astemizole, benzene, cadmium, carbamezipine, carbon tetrachloride (CCl4), ciprofibrate (cipro), clofibrate, cobalt chloride, corvastatin, cyclosporin A, diethylntrosamine, dimethylformamide, dimethylhydrazine (DMH), diquat, ethosuximide, etoposide, famotidine, fluconazole, gamfibrozil, ganciclovir, hexachloro-1,3-butediene (HCBD), HIV protease inhibitors, hydrazine, indomethacin, interleukin-6 (IL-6), ketoconazole, lead acetate (PbAc), lipopolysaccharide (LPS), mercury(II) chloride (HgCl₂), methanol, methapyrilene, methotrexate, metronidazole, miconazole, monocrotaline, nitric oxide, ondansetron, pentamidine, phenobarbital, phenylhydrazine (phenylhyrzn), phenytoin, pravastatin, propulsid, puromycin aminonucleoside (PAN), quinolones, simvastatin, sodium fluoride (NaF), statins, thioacetamide, tocainidine, tricyclic antidepressants, troglitazone, tumor necrosis factor α (TNFα), uranyl nitrate, valproic acid, vincristine, Wy-16,463, zidovudine (AZT), α-naphthyl isothiocyanate (ANIT), or β-naphthoflavone (BNF).

In another embodiment, the invention provides a method for analyzing small molecules. The analytic method can be mass spectroscopy (MS), HPLC, TLC, electrochemical analysis, refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), and Light Scattering analysis (LS). The analytic method can include two or more of the methods described above.

In another embodiment, at least 174 specific small molecules can be analyzed by mass spectroscopy and mass spectroscopy analysis is capable of being performed in at most 8.5 minutes. mass spectroscopy analysis can include staggering injections using a multiple column switching valve, which allows combinations of different column types into one injection. The concentration of each small molecule analyzed can be below the concentration of 10 ng/ml.

In another embodiment, the generation of small molecule efficacy, toxicity and disease profiles and signatures can include a computer system for tracking samples.

### BRIEF DESCRIPTION OF THE DRAWINGS

FIG. 1 is a schematic of the instrument for the targeted platform showing A.) three columns being routed to the spectrometer and B) four assay columns and the column switcher routed to the spectrometer.

FIG. 2 is a photograph of mass spectroscopy analysis showing serum data from hydrazine and vehicle treated rats (median-normalized, log peak area ratio).

FIG. 3 is a photograph of mass spectroscopy analysis showing time-course differences per treatment in serum data in 10 rat replicates

FIG. 4 is a photograph of mass spectroscopy analysis showing time-course differences per treatment in serum data in analytes where F>8 (16 out of 44).

FIG. 5 is a line graph showing hydrazine signature in serum data for 16 amino acid analytes.

FIG. 6 is a series of bar graphs showing Tylenol treatment and mock treatment in rats.

### DETAILED DESCRIPTION

The present invention is directed to methods for generating and using small molecule profiles and signatures. In a comparison of two groups of subjects with an identified difference, e.g., healthy and diseased subjects or treated and untreated subjects, the levels of expression of multiple small molecules will be different in predictive ways. The measurement of the levels of each of these small molecules in a sample from a biological source from a subject creates a small molecule profile. There will be differences in the levels of hundreds of small molecules between the two groups of subjects. Out of the hundreds of small molecules whose levels change between the two groups of subjects, there are a subset of small molecules whose changes are more relevant for either predicting disease, drug efficacy, or drug toxicity. This subset of small molecule changes is a small molecule signature.

The generation of small molecule profiles and signatures involves the analysis of large numbers of small molecules. One aspect of the present invention is the ability to analyze great numbers of pharmacologically relevant small molecules from a single subject in a small amount of time. A pharmacologically relevant small molecule is a small molecule which has been identified prior to analysis as one for which analysis results are desired. In this regard, before analyzing a sample for its small molecule profile, one would specifically identify a set of small molecules for obtaining analysis results.

One problem known in the art of small molecule profiling is the difficulty of analyzing hundreds of pharmacologically relevant small molecules in a sample from a single subject in a relatively short period of time. In the present invention, outlined in detail in Example 7, we have demonstrated that 174 specific small molecules have been analyzed in less than 9 minutes. The small molecules analyzed therein were specifically chosen prior to the analysis. Although it has been known that hundreds of small molecules can be analyzed in a relatively short time frame, this has only been shown with random small molecules, i.e., small molecules which had not been previously identified as those for which analysis was desired. The present invention demonstrates that it is possible to generate in a short time frame an analysis profile of greater than 100 small molecules which had been specifically identified prior to the undertaking of the analysis. In certain embodiments, the present invention can analyze greater than 100, more preferably greater than 150, and even more preferably greater than 170 specific small molecules in a short time frame. The short time frame can be less than 20 minutes, more preferably less than 10 minutes, and even more preferably less than 9 minutes.

In analyzing samples in order to generate small molecule profiles, a large number of samples and other reagents are involved. It is very important for the accuracy of profile and signature generation that each component of the analysis be tracked as to its position in the process. In this manner, a Laboratory Information Management System (LIMS) can be used to help track all of the components of the process. Accordingly, another aspect of the present invention is the use of a LIMS system in the generation of small molecule profiles and signatures. More details of the use of such a tracking system in the present invention are given in Example 6.

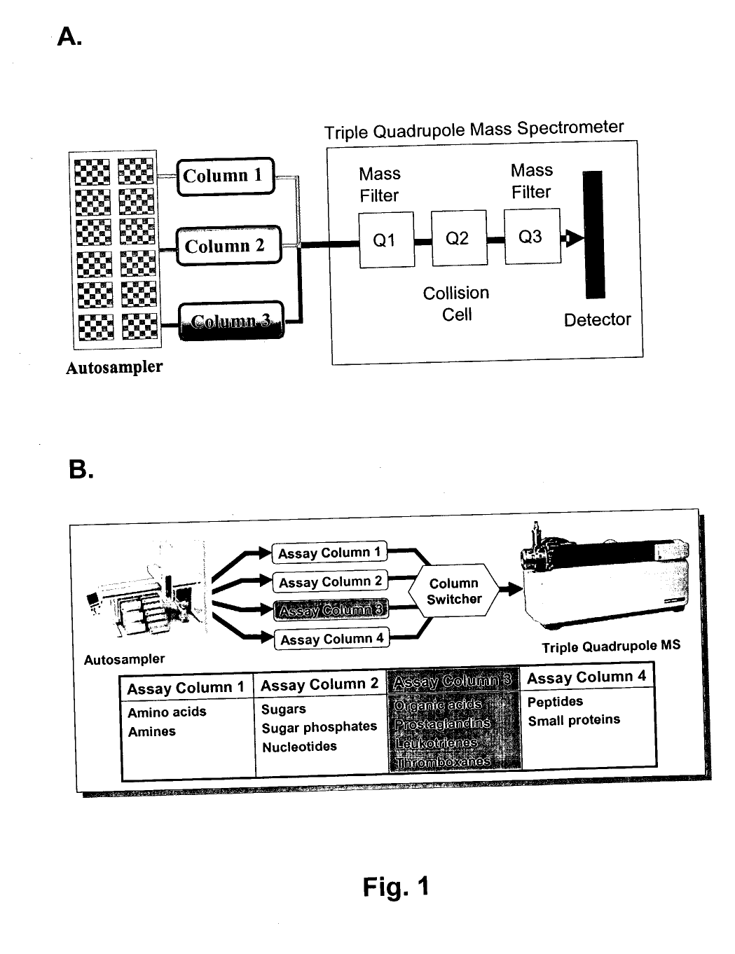

As described in more detail below, one embodiment of the present invention uses mass spectroscopy as a method for analyzing the small molecules contained in a biological source taken from a subject. In one embodiment, small molecules from a subject are separated through the use of column chromatography. Multiple columns are used, with each column designed to separate classes of compounds. In this format, there can be a column switching valve which allows for staggered injections into the multiple columns. This format is illustrated in FIGS. 1A and 1B. Additional details of the specifics of mass spectroscopy and the column switching valve are described in Example 4.

The present invention is also directed to methods of using small molecule profiles and signatures which have been generated using the method steps described above. Small molecule signatures can be utilized, inter alia, to generate an efficacy signature or a toxicity signature for a particular compound, or to generate a disease signature for a particular subject. Additionally, a database of such signatures can be generated. Such a database can be used to help identify the efficacy or toxicity of a compound by providing the ability to screen the signatures of unknown compounds in a high throughput manner against a database of signatures of known compounds. Each of an efficacy signature, a toxicity signature, and a disease signature is described briefly below and is described in more detail throughout the specification.

An efficacy signature can be used when screening compounds of potential new drugs to see if the particular small molecule signature generated by treatment with the potential new drug is predictive of a specific efficacy. For example, a drug or other compound with an unknown function can be used to treat subjects. A small molecule profile determining the levels of small molecules for each treated subject would then be generated. A similar profile would be generated for untreated control subjects. A comparison of the small molecule profiles from treated and untreated subjects allows a signature for that drug to be generated. The levels of small molecules present in the subjects can be determined in a variety of ways known to one of skill in the art, including the use of mass spectroscopy. Specific aspects of the mass spectroscopy are described in detail herein. The small molecule signature generated can then be compared to either a single signature of a compound with a known efficacy or to a database of signatures generated by drugs of specific classes of compounds. If the signature generated by treatment with the unknown compound is similar to signatures of a known class of compounds, then the unknown compound can be predicted to have an efficacy similar to the known class of compounds with a similar small molecule signature. In this way, an efficacy signature can be used to predict efficacy of unknown compounds.

A toxicity signature can be used to determine whether compounds of potential new drugs are safe or are toxic. For example, a potential new drug or other compound with an unknown toxicity can be used to treat subjects. A small molecule profile determining the levels of small molecules for each treated subject would then be generated. A similar profile would be generated for untreated control subjects. A comparison of the small molecule profiles from treated and untreated subjects allows a signature for that drug to be generated The levels of small molecules present in the subjects can be determined in a variety of ways known to one of skill in the art, including the use of mass spectroscopy. The small molecule signature generated can then be compared to either a single signature of a compound with a known toxicity or to a database of toxicity signatures generated by drugs of known toxicities. If the signature generated by treatment with the unknown compound is similar to a compound which is known to be non-toxic, then the unknown compound can be classified similarly as being non-toxic. In this way, a toxicity signature can be used to predict the toxicity or safety of unknown compounds.

In another embodiment of the present invention, a disease signature can be used for diagnosing a disease or disorder in a subject or for monitoring the progression or remission of a disease or disorder in a subject. For example, a biological source can be taken from a subject with an unknown disease or disorder. The levels of small molecules present in the subject can be determined in a variety of ways known to one of skill in the art, including the use of mass spectroscopy. A similar profile would be generated for a healthy control subject. A comparison of the small molecule profiles from diseased and healthy subjects allows a signature for that disease to be generated The small molecule signature generated can then be compared to either a single signature taken from a subject with a known disease or disorder or to a database of signatures taken from subjects with a variety of known diseases or disorders. If the signature generated by the subject with the unknown disease or disorder is similar to a signature taken from a subject with a known disease or disorder, then the subject can be diagnosed with a disease with a similar signature. In this way, a disease signature can be used to diagnose a disease or disorder in a subject.

Additionally, in another embodiment, the present invention includes a method of monitoring the progression or remission of a disease in a subject for the purpose of determining the effectiveness of the treatment. The method includes diagnosing a disease or disorder using the small molecule disease signature described above, and then periodically generating a small molecule disease profile from biological sources isolated from the subject during the course of medical treatment. A gradual loss of the small molecule disease signature from the subject indicates successful treatment of the disease.

The methods of generating small molecules profiles and signatures and the uses of such profiles and signatures are described in further detail below.

Small Molecule Profiles and Small Molecule Signatures

Small molecule profiles are an inventory of small molecules found in a given sample. In preferred embodiments, the sample may be obtained, as described herein, from a subject that has been either treated or untreated with a drug, agent or toxicant, or from a subject that has a particular disease state or is healthy.

Small molecule signatures are an inventory of the changes in levels of small molecules that are associated with a particular treatment or disease state that is made possible by comparing many small molecule profiles. In preferred embodiments, small molecule signatures for a particular treatment or disease are obtained by comparing multiple small molecule profiles for samples from similar subjects (e.g. treated with the same drug or agent, or having the same disease) with profiles from untreated or healthy subjects.

Small Molecule Signature Generation and Utilization

In a preferred embodiment, small molecule signatures are generated and utilized in two steps. The first step is the generation step where samples are obtained from a number of subjects selected from each of two groups—typically treated (with agent, drug or toxicant) and untreated, or diseased and healthy. The contrasting nature of the two groups enables a signature to be developed that describes the differences in the small molecule inventories associated with this contrast. Thus a signature can be developed for a particular disease, or for the efficacy or toxicity of a given class of drugs, agents or toxicants.

The second step is the utilization step where small molecule signatures are used to assess small molecule profiles from subjects treated with a drug, agent or toxicant of unknown efficacy or toxicity, or from subjects with unknown disease state. By comparing these small molecule profiles with a small molecule signature, it is possible to assign the subject as either healthy or diseased, or to describe the drug, agent or toxicant as having a particular efficacy or toxicity.

The language “small molecule signature” includes the inventory of the changes in levels of small molecules in tangible form within a targeted cell, tissue, organ, organism, or any derivative fraction thereof, e.g., cellular compartment, from a subject that is necessary and/or sufficient to provide information to a user for its intended use within the methods described herein. The “small molecule signature” is made possible by comparing many small molecule profiles. The inventory would include the quantity and/or type of small molecules present The ordinarily skilled artisan would know that the information which is necessary and/or sufficient would vary depending on the intended use of the “small molecule signature.”

The generation of small molecule profiles to be utilized in assembling small molecule signatures is described herein.

Small Molecule Efficacy Signature Generation and Utilization

In preferred embodiments, the invention is directed to a method of generating small molecule efficacy signatures and utilizing the small molecule efficacy signatures to determine the effect of an unknown agent or drug on a subject or biological source.

In one embodiment, the invention includes a method of generating a small molecule efficacy profile from a subject. The method includes isolating a biological source from the subject treated with a particular agent or drug. Further, the method includes extracting small molecules from the biological source isolated from the treated subject and analyzing the small molecules. The small molecule analysis results in the generation of a small molecule efficacy profile.

In another embodiment, the invention includes a method of generating a small molecule efficacy signature from a subject. The method includes obtaining the small molecule efficacy profile, as described above, and comparing that small molecule efficacy profile to a small molecule efficacy profile, from an untreated subject, generated from a similar biological source. Comparing the small molecule efficacy profiles results in the generation of a small molecule efficacy signature.

In another embodiment, the invention includes a method of determining the effectiveness of an agent or drug of unknown efficacy in a subject. The method includes obtaining the small molecule efficacy signature, as described above, and comparing the small molecule efficacy signature to a small molecule efficacy signature or a database of small molecule efficacy signatures generated from biological sources isolated from subjects treated with a range of known agents or drugs. Comparing the small molecule efficacy signature of the unknown drug with the efficacy signature or efficacy signatures in the database determines the effect of the unknown agent or drug on the subject and biological source.

In another embodiment, the invention includes a method of comparing small molecule efficacy profiles and signatures, by generating a data matrix including two or more analyte/sample values indicating small molecule abundance in the sample, log transforming the data matrix, normalizing the transforming data matrix which includes subtracting the median of all analyte/sample values from each analyte/sample value and performing variance analysis on the normalized data matrix, thereby generating small molecule efficacy profiles and signatures.

The biological source can include but is not limited to an organ, tissue, cell, cellular compartment, organelle, cerebrospinal fluid, synovial fluid, blood or urine. The biological source can be prokaryotic or eukaryotic. Preferably, the biological source is mammalian. More preferably, the biological source is human.

The small molecules can be analyzed by mass spectroscopy (MS), IPLC, TLC, electrochemical analysis, refractive index spectroscopy (RI), Ultra-Violet spectroscopy (UV), fluorescent analysis, radiochemical analysis, Near-InfraRed spectroscopy (Near-IR), Magnetic Resonance spectroscopy (NMR), Light Scattering analysis (LS). Preferably, the small molecules are analyzed by mass spectroscopy.

In another embodiment, at least 174 specific small molecules can be analyzed by mass spectroscopy and mass spectroscopy analysis is capable of being performed in at most 8.5 minutes. mass spectroscopy analysis can include staggering injections using a multiple column switching valve, which allows combinations of different column types into one injection. The concentration of each small molecule analyzed can be below the concentration of 20 ng/ml, more preferably below 15 ng/ml, and even more preferably below 10 ng/ml.

The short time frame can be less than 20 minutes, more preferably less than 10 minutes, and even more preferably less than 9 minutes.

In another embodiment, the generation of small molecule efficacy profiles and signatures can include a computer system for tracking samples.

The subject can include but is not limited to humans, dogs, cats, horses, cattle, sheep, pigs, llamas, gerbils, squirrels, goats, bears, chimpanzees, mice, rats and rabbits. Preferably the subject is human. The subject can be a healthy reference subject or the subject can suffer from a disease or disorder. The disease or disorder can include but is not limited to non-insulin-dependent diabetes (NIDDM), rheumatoid arthritis or inflammation (RA/I), immunological disorder, metabolic disorder, cardiovascular disorder, neurological disorder, oncological disorder, and viral disorder.

The agent or drug can include but is not limited to Chlorpropamide, Tolbuamide, Tolazamide, Acetohexamide, Glyburide, Glipizide, Glimepiride, Pioglitazone, Rosiglitazone, Metformin, Acarbose (Precose), Miglitol (Glycet), Repaglinide (Prandin), Aspirin, Acetaminophen, Ibuprofen, Indomethacin, Peroxicam, Tometin, Rofecoxib, Celecoxib, Valdecoxib, Methotrexate, and Dexamethasone.

Small Molecule Toxicity Signature Generation and Utilization

In preferred embodiments, the invention is directed to a method of generating small molecule toxicity signatures and utilizing the small molecule toxicity signatures to determine the effect of an agent of unknown toxicity on a subject or biological source.

In one embodiment, the invention includes a method of generating a small molecule toxicity profile from a subject. The method includes isolating a biological source from the subject treated with a particular toxicant. Further, the method includes extracting small molecules from the biological source isolated from the treated subject and analyzing the small molecules. The small molecule analysis results in the generation of a small molecule toxicity profile.

In another embodiment, the invention includes a method of generating a small molecule toxicity signature from a subject. The method includes obtaining the small molecule toxicity profile, as described above, and comparing that small molecule toxicity profile to a small molecule toxicity profile, from an untreated subject, generated from a similar biological source. Comparing the small molecule toxicity profiles results in the generation of a small molecule toxicity signature.

In another embodiment, the invention includes a method of determining the toxicity of a drug or agent in a subject. The method includes obtaining the small molecule toxicity signature, as described above, and comparing the small molecule toxicity signature to a small molecule toxicity signature or a database of small molecule toxicity profiles generated from biological sources isolated from subjects treated with a range of known toxicants. Comparing the small molecule toxicity signature with the small molecule toxicity signature or the small molecule toxicity signature database determines the toxicity of the unknown drug or agent on the subject and biological source.

In another embodiment, the invention includes a method of comparing small molecule toxicity profiles and signatures, by generating a data matrix including two or more analyte/sample values indicating small molecule abundance in the sample, log transforming the data matrix, normalizing the transforming data matrix which includes subtracting the median of all analyte/sample values from each analyte/sample value and performing variance analysis on the normalized data matrix, thereby generating small molecule toxicity profiles and signatures.

The biological source can include but is not limited to an organ, tissue, cell, cellular compartment, organelle, cerebrospinal fluid, synovial fluid, blood or urine. The biological source can be prokaryotic or eukaryotic. Preferably, the biological source is mammalian. More preferably, the biological source is human.

The small molecules can be analyzed by mass spectroscopy (MS), HPLC, TLC, electrochemical analysis, refractive index spectroscopy (RI), Ultra-Violet spectroscopy (UV), fluorescent analysis, radiochemical analysis, Near-InfraRed spectroscopy (Near-IR), Magnetic Resonance spectroscopy (NMR), Light Scattering analysis (LS). Preferably, the small molecules are analyzed by mass spectroscopy.

In another embodiment, at least 174 specific small molecules can be analyzed by mass spectroscopy and mass spectroscopy analysis is capable of being performed in at most 8.5 minutes. mass spectroscopy analysis can include staggering injections using a multiple column switching valve, which allows combinations of different column types into one injection. The concentration of each small molecule analyzed can be below the concentration of 20 ng/ml, more preferably below 15 ng/ml, and even more preferably below 10 ng/ml.

In another embodiment, the generation of small molecule toxicity profiles and signatures can include a computer system for tracking samples.

The subject can include but is not limited to humans, dogs, cats, horses, cattle, sheep, pigs, llamas, gerbils, squirrels, goats, bears, chimpanzees, mice, rats and rabbits. Preferably the subject is human. The subject can be a healthy reference subject or the subject can suffer from a disease or disorder. The disease or disorder can include but is not limited to non-insulin-dependent diabetes (NIDDM), rheumatoid arthritis or inflammation (RA/I), immunological disorder, metabolic disorder, cardiovascular disorder, neurological disorder, oncological disorder, and viral disorder.

The toxicant can include but is not limited to 2,2′,4,4′,5,5′-hexachlorobiphenyl (PCB-153), 2,3,7,8-tetrachlorodibenzo-p-dioxin (TCDD), 2-bromoethylamine (BEA), 3-methylcholanthrene, 4-aminophenol (PAP), acetaminophen, adriamycin, allyl alcohol, amiodarone, amphotericin B, Aroclor 1254, Aroclor 1260, arsenic, aspirin, astemizole, benzene, cadmium, carbamezipine, carbon tetrachloride (CCl4), ciprofibrate (cipro), clofibrate, cobalt chloride, corvastatin, cyclosporin A, diethylntrosamine, dimethylformamide, dimethylhydrazine (DMH), diquat, ethosuximide, etoposide, famotidine, fluconazole, gamfibrozil, ganciclovir, hexachloro-1,3-butediene (HCBD), HIV protease inhibitors, hydrazine, indomethacin, interleukin-6 (IL-6), ketoconazole, lead acetate (PbAc), lipopolysaccharide (LPS), mercury(II) chloride (HgCl₂), methanol, methapyrilene, methotrexate, metronidazole, miconazole, monocrotaline, nitric oxide, ondansetron, pentamidine, phenobarbital, phenylhydrazine (phenylhyrzn), phenytoin, pravastatin, propulsid, puromycin aminonucleoside (PAN), quinolones, simvastatin, sodium fluoride (NaF), statins, thioacetamide, tocainidine, tricyclic antidepressants, troglitazone, tumor necrosis factor α (TNFα), uranyl nitrate, valproic acid, vincristine, Wy-16,463, zidovudine (AZT), α-naphthyl isothiocyanate (ANIT), and β-naphthoflavone (BNF).

Small Molecule Disease Signature Generation and Utilization

In preferred embodiments, the invention is directed to a method of generating small molecule disease signatures and utilizing the small molecule disease signatures to diagnose an unknown disease or disorder in a subject.

In one embodiment, the invention includes a method of generating a small molecule disease profile from a subject suffering from a disease or disorder. The method includes isolating a biological source from a diseased subject. Further, the method includes extracting small molecules from the biological source isolated from the diseased subject and analyzing the small molecules. The small molecule analysis results in the generation of a small molecule disease profile.

In another embodiment, the invention includes a method of generating a small molecule disease signature from a subject. The method includes obtaining the small molecule disease profile, as described above, and comparing that small molecule disease profile to a small molecule disease profile, from a healthy, non-diseased subject, generated from a similar biological source. Comparing the small molecule disease profiles results in the generation of a small molecule disease signature.

In another embodiment, the invention includes a method of diagnosing a disease or disorder in a subject. The method includes obtaining the small molecule disease signature, as described above, and comparing the small molecule disease signature to a small molecule disease signature or a database of small molecule disease signatures generated from biological sources isolated from subjects with a range of known diseases or disorders. Comparing the small molecule disease signature with the small molecule disease signature or the small molecule disease signature database diagnoses the unknown disease or disorder in the subject.

In another embodiment, the invention includes a method of comparing small molecule disease profiles and signatures, by generating a data matrix including two or more analyte/sample values indicating small molecule abundance in the sample, log transforming the data matrix, normalizing the transforming data matrix which includes subtracting the median of all analyte/sample values from each analyte/sample value and performing variance analysis on the normalized data matrix, thereby generating small molecule disease profiles and signatures.

In another embodiment, the invention includes a method of monitoring the progression or remission of a disease in a subject for the purpose of determining the effectiveness of the treatment. The method includes diagnosing a disease using the small molecule disease signature described above, and then periodically generating a small molecule disease profile from biological sources isolated from the subject during the course of medical treatment. A gradual loss of the small molecule disease signature from the subject indicates successful treatment of the disease.

The biological source can include but is not limited to an organ, tissue, cell, cellular compartment, organelle, cerebrospinal fluid, synovial fluid, blood or urine. The biological source can be prokaryotic or eukaryotic. Preferably, the biological source is mammalian. More preferably, the biological source is human.

The small molecules can be analyzed by mass spectroscopy (MS), HPLC, TLC, electrochemical analysis, refractive index spectroscopy (RI), Ultra-Violet spectroscopy (UV), fluorescent analysis, radiochemical analysis, Near-InfraRed spectroscopy (Near-IR), Magnetic Resonance spectroscopy (NMR), Light Scattering analysis (LS). Preferably, the small molecules are analyzed by mass spectroscopy.

In another embodiment, at least 174 specific small molecules can be analyzed by mass spectroscopy and mass spectroscopy analysis is capable of being performed in at most 8.5 minutes. mass spectroscopy analysis can include staggering injections using a multiple column switching valve, which allows combinations of different column types into one injection. The concentration of each small molecule analyzed can be below the concentration of 20 ng/ml, more preferably below 15 ng/ml, and even more preferably below 10 ng/ml.

In another embodiment, the generation of small molecule disease profiles and signatures can include a computer system for tracking samples.

The subject can include but is not limited to humans, dogs, cats, horses, cattle, sheep, pigs, llamas, gerbils, squirrels, goats, bears, chimpanzees, mice, rats and rabbits. Preferably the subject is human. The subject can be a healthy reference subject or the subject can suffer from a disease or disorder. The disease or disorder can include but is not limited to non-insulin-dependent diabetes (NIDDM), rheumatoid arthritis or inflammation (RA/I), immunological disorder, metabolic disorder, cardiovascular disorder, neurological disorder, oncological disorder, and viral disorder.

Small Molecule Profiles of Biological Samples

The invention pertains, at least in part, to the generation of small molecule profiles of samples (e.g. biological), tissues, cells, and cellular compartments. Small molecule profiles “fingerprint” the cell or cellular compartment and identify the presence, absence or relative quantity of small molecules. The small molecule profiles of the cells or cellular compartments may be obtained through, for example, a single technique or a combination of techniques for separating and/or identifying small molecules known in the art. Examples of separation and analytical techniques which can be used to separate and identify the compounds of the small molecule signatures include, but are not limited to, mass spectroscopy (MS), HPLC, TLC, electrochemical analysis, refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), Light Scattering analysis (LS) and other methods known in the art. Preferably, the methods of the invention detect both electrically neutral as well as electrochemically active compounds. More preferably, the separation and analytical technique is MS. Detection and analytical techniques can be arranged in parallel to optimize the number of molecules identified.

The term “sample” includes tissue or cellular extracts or any biological material from which a small molecule profile of the extract can be obtained. In one embodiment, biological sample sources include cell lines, mammalian tissue (e.g. human, rat, etc) or any other tissue or cellular source. In one embodiment, the samples are substantially free of macromolecules (e.g., large proteins and polynucleotides with molecular weights of greater than 10,000). The sample may be obtained from the entire tissue, entire cell or from specific cellular compartments. Examples of specific cellular compartments include the cytoplasm, the mitochondria, the Golgi apparatus, the endoplasmic reticulum, the nucleus, the chloroplasts, the cytosol, etc. The term “samples” includes both isolated small molecules and mixtures of small molecules.

The term “cells” includes prokaryotic cells, eukaryotic cells, yeast cells, bacterial cells, plant cells, animal cells, such as, reptilian cells, bird cells, fish cells, mammalian cells. Permanent and non-permanent cell lines are included in the invention. In one embodiment, non-permanent cell lines are used to generate hepatic, or other organ, toxicity profiles. Preferred cells include those derived from humans, dogs, cats, horses, cattle, sheep, pigs, llamas, gerbils, squirrels, goats, bears, chimpanzees, mice, rats, rabbits, etc. The term cells includes transgenic cells from cultures or from transgenic organisms. The cells may be from a specific tissue, body fluid, organ (e.g., brain tissue, nervous tissue, muscle tissue, retina tissue, kidney tissue, liver tissue, etc.), or any derivative fraction thereof. The term includes healthy cells, transgenic cells, cells affected by internal or exterior stimuli, cells suffering from a disease state or a disorder, cells undergoing transition (e.g., mitosis, meiosis, apoptosis, etc.), etc. In some embodiments, cell lines include but are not limited to, NIH/3T3, CHO-K1, CaCo2, LLC-PK1, HK2, HepG2, MC/9 and BRL3A, however any cell line may be used.

Cell culture techniques used in propagation, maintenance, storage of cells are those commonly used in the art, such as those described in “Culture of Animal Cells (4^{th} ed.)” (R. Ian Freshney; Wiley-Liss, New York; 2000) or in “Basic Cell Culture Protocols (2^{nd} ed.)” (Pollard, J W and Walker, J M, eds.; Humana Press, Totowa, N.J., 1997.)

In a further embodiment, the samples are obtained from a specific cellular compartment. The term “cellular compartment” includes organelles (such as mitochondria, Golgi apparatus, centrioles, chloroplasts), the nucleus, the cytoplasm (optionally including the organelles), and other cellular regions capable of being isolated. In one embodiment, the cellular compartment is the entire cell or entire tissue.

The analysis of a particular cellular compartment has many advantages over analysis of tissues, whole cells, whole cell lysates, body fluids, etc. For example, often the mechanism of action of a drug, an agent, a toxic compound, etc. is directed to a specific cellular function, such as, for example, the electron transport chain in the mitochondria, nucleic acid replication in the nucleus, etc. By isolating the specific cellular compartment or organelle (e.g., mitochondria, nuclei, Golgi apparatus, endoplasmic reticulum, ribosomes, etc.), it is possible to narrow the focus of the profile to small molecules involved in the relevant pathway. By narrowing the scope of the study to the particular organelle, researchers will be able to study the pathway of interest in more detail without irrelevant molecules present in interstitial fluid, blood, spinal fluid, saliva, etc.

The term “small molecules” includes organic and inorganic molecules which are present in the tissue, fluid, cell, cellular compartment, or organelle. The term does not include large macromolecules, such as large proteins (e.g., proteins with molecular weights over 2,000, 3,000, 4,000, 5,000, 6,000, 7,000, 8,000, 9,000, 10,000, 20,000, or 30,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of small molecules include sugars, fatty acids, amino acids, nucleotides, intermediates formed during cellular processes, and other small molecules found within the cell. In one embodiment, the small molecules of the invention are isolated.

The term “metabolome” includes all of the small molecules present in a given organism. The metabolome includes both metabolites as well as products of catabolism. In one embodiment, the invention pertains to a small molecule profile of the entire metabolome of a species. In another embodiment, the invention pertains to a computer database (as described below) of the entire metabolome of a species, e.g., an animal, e.g., a mammal, e.g., a mouse, rat, rabbit, pig, cow, horse, dog, cat, bear, monkey, and, preferably, a human. In another embodiment, the invention pertains to a small molecule library of the entire metabolome of an organism (as described below), e.g., a mammal, e.g., a mouse, rat, rabbit, pig, cow, horse, dog, cat, bear, monkey, and, preferably, a human.

The language “small molecule profile” includes the inventory of small molecules in tangible form within a targeted cell, tissue, organ, organism, or any derivative fraction thereof, e.g., cellular compartment, that is necessary and/or sufficient to provide information to a user for its intended use within the methods described herein. The inventory would include the quantity and/or type of small molecules present. The ordinarily skilled artisan would know that the information which is necessary and/or sufficient would vary depending on the intended use of the “small molecule profile.” For example, the “small molecule profile,” can be determined using a single technique for an intended use but may require the use of several different techniques for another intended use depending on such factors as the disease state involved, the types of small molecules present in a particular targeted cellular compartment, the cellular compartment being assayed per se. etc. In a preferred embodiment, the “small molecule profile” is utilized to generate a small molecule signature to assess a particular disease state or the efficacy or toxicity of a particular class of drugs, agents or toxicants.

In a preferred embodiment, the small molecule profile may be an efficacy profile, toxicity profile or disease profile. An efficacy profile can compare a normal sample with or without drug/agent treatment or can compare a diseased sample with or without drug/agent treatment. A toxicity profile can compare a normal sample with or without a known toxicant. A disease profile can compare a normal sample and a disease sample. These profiles can be utilized to generate a toxicity signature, toxicity signature or disease signature.

The relevant information in a small molecule profile also may vary depending on the intended use of the compiled information, e.g. spectra. For example for some intended uses, the amounts of a particular small molecule or a particular class of small molecules may be relevant, but for other uses the distribution of types of small molecules may be relevant.

The ordinarily skilled artisan would be able to determine the appropriate small molecule profiles for each method described herein by comparing small molecule profiles from diseased and/or test subjects with standard and/or healthy subjects. These comparisons can be made by individuals, e.g., visually, or can be made using software designed to make such comparisons, e.g., a software program may provide a secondary output which provides useful information to a user. For example, a software program can be used to confirm a profile or can be used to provide a readout when a comparison between profiles is not possible with a “naked eye”. The selection of an appropriate software program, e.g., a pattern recognition software program, is within the ordinary skill of the art. An example of such a program is Pirouette. It should be noted that the comparison of the profiles could be done both quantitatively and qualitatively.

The small molecule profiles can be obtained from an organism suffering from a disease state, genetic alteration, a tissue or organism treated with a test drug or agent, or any of the models discussed in more detail below. In one embodiment, the small molecule profile of an organism is determined by using HPLC (Kristal, et al. Anal. Biochem. 263:18-25 (1998)), thin layer chromatography (TLC), or electrochemical separation techniques (see, WO 99/27361, WO 92/13273, U.S. Pat. No. 5,290,420, U.S. Pat No. 5,284,567, U.S. Pat No. 5,104,639, U.S. Pat No. 4,863,873, and U.S. Pat. No. 32,920). Other techniques for determining the presence of small molecules or determining the identity of small molecules of the cell are also included, such as refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), Light Scattering analysis (LS) and other methods known in the art. In a preferred embodiment, the small molecule profile is determined by mass spectroscopy (MS).

In one embodiment, the invention pertains to small molecule profiles generated by several methods, e.g., mass spectroscopy (MS), HPLC, TLC, electrochemical analysis, refractive index spectroscopy (RI), Ultra-Violet spectroscopy (UV), fluorescent analysis, radiochemical analysis, Near-InfraRed spectroscopy (Near-IR), Magnetic Resonance spectroscopy (NMR), Light Scattering analysis (LS) and other methods known in the art.

The methods of the invention have several advantages over methods which rely only on a single mode of analysis, such as electrochermical separation. While electrochemical separation works only for “electrochemically” active compounds, it does not effectively separate neutral molecules. The invention here relates to the use in tandem and in parallel of a multitude of these detectors. This will result in the identification of a more comprehensive database. The detectors are usually attached to the HPLC columns where they can detect and emit a response due to the eluting sample and subsequently signal a peak on a chromatogram. The bandwidth and height of the peaks may usually be adjusted using the coarse and fine tuning controls and the detection and sensitivity parameters may also be controlled. Many detectors can be used with the HPLC. Some detectors which can be used in the methods of the invention include, but are not limited to, Mass Spectroscopy (MS), Refractive Index (RI), Ultra-Violet (UV), Fluorescent, Radiochemical, Electrochemical, Near-InfraRed (Near-IR), Nuclear Magnetic Resonance (NMR), Light Scattering (LS) among others.

The methods of the invention can be used to detect both electrochemically active molecules as well as electrochemically neutral molecules. In a further embodiment, the invention pertains to methods which detect about 50% or more, about 60% or more, about 70% or more, about 75% or more, about 77.5% or more, about 80% or more, about 82.5% or more, about 85% or more, about 86% or more, about 87% or more, about 88% or more, about 89% or more, about 90% or more, about 91% or more, about 92% or more, about 93% or more, about 94% or more, about 95% or more, about 96% or more, about 97% or more, about 98% or more, about 99% or more of the small molecules of a cell or cellular compartment (e.g., mitochondria, chloroplast, endoplasmic reticulum, nuclei, Golgi apparatus, cytosol, etc.).

In one embodiment, HPLC columns equipped with coulometric array technology can be used to analyze the samples, separate the compounds, and/or create a small molecule profile of the samples. Such HPLC columns have been used extensively in the past for serum, urine and tissue analysis and are suitable for small molecule analysis (Acworth et al., 300; Beal et al., J Neurochem. 55, 1327-1339, 1990; Matson et al., Life Sci. 41, 905-908, 1987; Matson et al., Basic, Clinical and Therapeutic Aspects of Alzheimer's and Parkinson's Diseases, vol II, pp. 513-516, Plenum, New York 1990; LeWitt et al., Neurology 42, 2111-2117, 1992; Milbury et al., J. Wildlife Manag., 1998; Ogawa et al., Neurology 42, 1702-1706, 1992; Beal et al., J. Neurol. Sci 108, 80-87, 1992, Matson et al., Clin. Chem. 30, 1477-1488, 1984; Milbury et al., Coulometric Electrode Array Detectors for HPLC, pp. 125-141, VSP International Science Publication; Acworth et al., Am. Lab 28, 33-38, 1996). HPLC columns equipped with coulometric arrays have been used for the simultaneous analysis of the majority of low-molecule weight, redox-active compounds in mitochondria. (Anal. Biochem. 263, 18-25, 1998).

For the detection and characterization of the small molecules in an effort to create a comprehensive small molecule profiles, a multitude of detection methods can be used. These methods are described in more detail below.

Mass Spectroscopy (MS) Detectors: The sample compound or molecule is ionized, it is passed through a mass analyzer, and the ion current is detected. There are various methods for ionization. Examples of these methods of ionization include electron impact (EI) where an electric current or beam created under high electric potential is used to ionize the sample migrating off the column, chemical ionization utilizes ionized gas to remove electrons from the compounds eluting from the column; and fast atom bombardment where Xenon atoms are propelled at high speed in order to ionize the eluents from the column. Mass Spectroscopy is described in detail below.

Pyrolysis Mass Spectrometry: Pyrolysis is the thermal degradation of complex material in an inert atmosphere or vacuum. It causes molecules to cleave at their weakest points to produce smaller, volatile fragments called pyrolysate (Irwin 1982). Curie-point pyrolysis is a particularly reproducible and straightforward version of the technique, in which the sample, dried onto an appropriate metal is rapidly heated to the Curie-point of the metal. A mass spectrometer can then be used to separate the components of the pyrolysate on the basis of their mass-to-charge ratio to produce a pyrolysis mass spectrum (Meuzelaar et al 1982) which can then be used as a “chemical profile” or fingerprint of the complex material analyzed. The combined technique is known as pyrolysis mass spectrometry (PyMS).

Nuclear Magnetic resonance (NMR) Detectors: Certain nuclei with odd-numbered masses, including H and ¹³C, spin about an axis in a random fashion. When they are placed between poles of a strong magnet, the spins are aligned either parallel or anti-parallel to the magnetic field, with parallel orientation favored since it is slightly lower energy. The nuclei are then irradiated with electromagnetic radiation which is absorbed and places the parallel nuclei into a higher energy state where they become in resonance with radiation. Different spectra will be produced depending on the location of the H or ¹³C and on adjacent molecules or elements in the compound because all nuclei in molecules are surrounded by electron clouds which change the encompassing magnetic field and thereby alter the absorption frequency.

Refractive Index (RI): In this method, detectors measure the ability of samples to bend or refract light. This property for each compound is called refractive index. For most RI detectors, light proceeds through a bi-modular flow to a photodetector. One channel of the flow-cell directs the mobile phase passing through the column while the other directs only the other directs only the mobile phase. Detection occurs when the light is bent due to samples eluting from the column, and is read as a disparity between the two channels. Laser based RI detectors have also become available.

Ultra-violet (UV) Detectors: In this method, detectors measure the ability of a sample to absorb light. This could be accomplished at a fixed wavelength usually 254 nm, or at variable wavelengths where one wavelength is measured at a time and a wide range is covered, alternatively Diode Array are capable of measuring a spectrum of wavelengths simultaneously. Sensitivity is in the 10⁻⁸ to 10⁻⁹ gm/ml range. Laser based absorbance or Fourier Transform methods have also been developed.

Fluorescent Detectors: This method measures the ability of a compound to absorb then re-emit light at given wavelengths. Each compound has a characteristic fluorescence. Each compound has a characteristic fluorescence. The excitation source passes through the flow-cell to a photodetector while a monochromator measures the emission wavelengths. Sensitivity is in the 10⁻⁹ to 10⁻¹¹ gm/ml. Laser based fluorescence detectors are also available.

Radiochemical Detection: This method involves the use of radiolabeled material, for example, tritium (³H) or carbon 14 (¹⁴C). It operates by detection of fluorescence associated with beta-particle ionization, and it is most popular in metabolite research. The detector types include homogeneous method where addition of scintillation fluid to column effluent causes fluorescence, or heterogeneous detection where lithium silicate and fluorescence by caused by beta-particle emission interact with the detector cell. Sensitivity is 10⁻⁹ to 10⁻¹⁰ gm/ml.

Electrochemical Detection: Detectors measure compounds that undergo oxidation or reduction reactions. Usually accomplished by measuring gains or loss of electrons from migration samples as they pass between electrodes at a given difference in electrical potential. Sensitivity of 10⁻¹² to 10⁻¹³ gms/ml.

Light Scattering (LS) Detectors: This method involves a source which emits a parallel beam of light. The beam of light strikes particles in solution, and some light is then reflected, absorbed, transmitted, or scattered. Two forms of LS detection may be used to measure transmission and scattering.

Nephelometry, defined as the measurement of light scattered by a particular solution. This method enables the detection of the portion of light scattered at a multitude of angles. The sensitivity depends on the absence of background light or scatter since the detection occurs at a black or null background. Turbidimetry, defined as the measure of the reduction of light transmitted due to particles in solution. It measures the light scatter as a decrease in the light that is transmitted through particulate solution. Therefore, it quantifies the residual light transmitted. Sensitivity of this method depends on the sensitivity of the machine employed, which can range from a simple spectrophotometer to a sophisticated discrete analyzer. Thus, the measurement of a decrease in transmitted light from a large signal of transmitted light is limited to the photometric accuracy and limitations of the instrument employed.

Near Infrared scattering detectors operate by scanning compounds in a spectrum from 700-1100 nm. Stretching and bending vibrations of particular chemical bonds in each molecule are detected at certain wavelengths. This is a fast growing method which offers several advantages; speed, simplicity of preparation of sample, multiple analyses from single spectrum and nonconsumption of the sample (McClure, 1994).

Fourier Transform Infrared Spectroscopy (FT-IR): This method measures dominantly vibrations of functional groups and highly polar bonds. The generated fingerprints are made up of the vibrational features of all the sample components (Griffiths 1986). FT-IR spectrometers record the interaction of IR radiation with experimental samples, measuring the frequencies at which the sample absorbs the radiation and the intensities of the absorptions. Determining these frequencies allows identification of the samples chemical makeup, since chemical functional groups are known to absorb light at specific frequencies. Both quantitative and qualitative analysis are possible using the FT-IR detection method.

Dispersive Raman Spectroscopy: Dispersive Raman Spectroscopy is a vibrational profile of a molecule or complex system. The origin of dispersive raman spectroscopy lies in the inelastic collisions between the molecules composing say the liquid and photons, which are the particles of light composing a light beam. The collision between the molecules and the photons leads to an exchange of energy with consequent change in energy and hence wavelength of the photon.

To create a small molecule profile, organs, tissues, cells, cellular compartments, or organelles are homogenized in standard ways know for those skilled in the art. Different fractionation procedures can be used to enrich the fractions for small molecules. An example fractionation procedure is described herein. The small molecules obtained will then be passed over several fractionation columns. The fractionation columns will employ a variety of detectors used in tandem or parallel to generate the small molecule signature for the organ, cell, cellular compartment, or organelle.

For example, to generate a small molecule profile of water-soluble molecules, the cell, cellular compartment, or organelle extracts could be fractionated on HPLC columns with a water-soluble array. The water-soluble small molecules can then be detected using fluorescence or UV detectors to generate the small molecule signatures. Alternatively, electrochemical detectors can be used with diads to pick up redox active compounds and the absorbance of active compounds. For generating detecting non water-soluble molecules, hydrophobic columns can also be used to generate small molecule signatures. In addition, gas chromatography combined with mass spectroscopy, liquid chromatography combined with mass spectroscopy, MALDI combined with mass spectroscopy, ion spray spectroscopy combined with mass spectroscopy, capillary electrophoresis, NMR and IR detection are among the many other combinations of separation and detection tools which can be used to generate small molecule signatures.

These small molecule profiles will be able to define and characterize organs, tissues, cells, cellular compartments, and organelles by their small molecule content in both health and disease states. The information generated by the small molecule profiles will be both qualitative and quantitative. In a preferred embodiment, these small molecule profiles can be utilized to generate small molecule signatures to assess, define and characterize a particular disease state or the efficacy or toxicity of a particular class of drugs, agents or toxicants.

Methods of Identification of Disease-relevant Small Molecules

In another embodiment, the invention includes a method of identifying disease-relevant small molecules and the generation of “disease signatures”. The method includes generating small molecule signatures from many small molecule profiles of tissues, cells, fluids, cellular compartments, or organelles from diseased subjects and comparing that to a standard signature generated from many small molecule profiles of a healthy tissue, cell, fluid, cellular compartment, or organelle. The method also involves identifying the small molecules which are present in aberrant amounts in the diseased small molecule signature. The small molecules present in aberrant amounts in the diseased cells are “disease-relevant small molecules.”

The language “disease-relevant small molecules” includes both small molecules present in aberrant amount in diseased small molecule profiles and signatures and, in addition, small molecules which are potentially involved in disease initiation, progression or prediction. The term also includes small molecules which are identified using the assays for particular diseases given below, as well as, compounds which are identified as being associated with particular genes of interest, also given below. The term also may include small molecules which when modulated, result in the lessening or curing of at least one symptom of a disease. The disease relevant small molecules are ideal drug candidates in screening assays.

For example, identified disease relevant small molecules may be screened using in vitro or in vivo assays known in the art to determine biological activity. The biological activity of disease relevant small molecules can also be pinpointed by using screening assays against protein targets which have been implicated in the disease state. In another embodiment, the biological activity of disease relevant small molecules can be determined using cell-based assays, e.g., tumor cell assays (Lillie et al. Cancer Res. 53(13):3172-8 (1993)). The disease relevant small molecules can also be tested for neuronal protection activity by exposing primary or cultured neurons to the compounds and toxic agents, such as glutamate, and identifying the compounds which protect the neurons from death. Animal models can also be used to further identify the biological activity of disease relevant small molecules. For example, animal models of Huntington's Disease, Parkinson's disease, and ALS can be used to identify small molecules useful as neuroprotective agents. (Kilvenyi, Nature Med. 5:347-350 (1999); Mathews et al, Experimental Neurology 157:142-149 (1999)).

Several animal models are contemplated. Generation of disease-specific profiles and signatures requires the comparison of signatures of the diseased state to signatures of the non-diseased state in rats of the same genetic background. In preferred embodiments, rat models are utilized for generating disease profiles and signatures. In one embodiment, rat experimental systems are used for two major disease classes, non-insulin dependent diabetes mellitus (NIDDM) and Rheumatoid arthritis/inflammation (RA/I):

NIDDM: Several rat strains are genetically predisposed to diabetes, including but not limited to, Zucker Diabetic Fatty (Zucker Fatty (fa/fa) selectively bred for hyperglycemia and glucose intolerance), Wistar Diabetic Fatty (Crossed Zucker fatty (fa/fa*) rat with carbohydrate intolerant, lean Wistar Kyoto), SHHF, Spontaneous Hypertension Heart Failure (Crossed “Koletsky obese” rats (cp/cp*) to SHRIN (spontaneously hypertensive) rats), and GK, Goto-Kakizaki (Selectively bred for high glucose levels from Wistar background).

RA/I: Rheumatoid arthritis (RA) and inflammation are experimentally induced in Sprague-Dawley rats using several methods, including but not limited to, injection of peptidoglycan-polysaccharide into joint, immunization with type II collagen, immunization with complete Freund's adjuvant, and injection of carrageenin into a subcutaneous air pouch.

In a further embodiment, the disease relevant small molecules can be chemically modified to further enhance their pharmaceutical or nutriceutical properties.

The term “disease” or “disease state” includes all disease which result or could potentially cause a change of the small molecule profile or signature of a cell, cellular compartment, or organelle in an organism afflicted with said disease. Examples of diseases include, but are not limited to, metabolic diseases (e.g., obesity, cachexia, diabetes, anorexia, etc.), cardiovascular diseases (e.g., atherosclerosis, ischemia/reperfusion, hypertension, restenosis, arterial inflammation, etc.), immunological disorders (e.g., chronic inflammatory diseases and disorders, such as Crohn's disease, reactive arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease, sarcoidosis, atopic conditions, such as asthma and allergy, including allergic rhinitis, gastrointestinal allergies, including food allergies, eosinophilia, conjunctivitis, glomerular nephritis, certain pathogen susceptibilities such as helminthic (e.g., leishmaniasis) and certain viral infections, including HIV, and bacterial infections, including tuberculosis and lepromatous leprosy, etc.), nervous system disorders (e.g., neuropathies, Alzheimer disease, Parkinson's disease, Huntington's disease, amyotropic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, peripheral nervous system diseases and mental disorders such as depression and schizophrenia, etc.), oncological disorders (e.g., leukemia, brain cancer, pancreatic cancer, prostate cancer, liver cancer, stomach cancer, colon cancer, throat cancer, breast cancer, ovarian cancer, skin cancer, melanoma, etc.). The term also includes disorders which result from oxidative stress.

The language “aberrant levels” includes any level, amount, or concentration of a small molecule in a cell, cellular compartment, or organelle which is different from the level of the small molecule of a standard sample. Aberrant levels and aberrant amounts are used interchangeable throughout the specification.

The language “standard signature” includes the comparison of many standard profiles. The term “standard profile” includes profiles derived from healthy cells, advantageously from a similar origin as the source. In one embodiment, the standard profile is an average of many samples of a certain cell type and/or a certain cellular compartment. In another embodiment, the standard profile may be derived from a patient prior to the onset of the disease state or from cells not affected by the disease state. Or, in another embodiment the standard profile can be an average of the profiles obtained from numerous sources, e.g., the standard profile may be an average of small molecule profiles obtained from two or more subjects. The standard profile can be a small molecule profile of a certain cellular compartment or from a certain subset of cells. In one embodiment, the invention pertains to the standard profile of healthy cells. Advantageously, the small molecules with aberrant levels in the sample are identified, e.g., mass spectroscopy (MS), HPLC, TLC, electrochemical analysis, refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), Light Scattering analysis (LS) and other methods known in the art. In one embodiment, the small molecule profile of the sample, cell, or cellular compartment, is compared to the standard profile by subtracting one profile from the other. The compounds which are present in aberrant amounts can then be used in drug design to identify deregulated cellular components. Standard profiles can also be made of the effects of certain agents (e.g., drugs, therapeutic agents, toxins, etc.) on both healthy and diseased cells (e.g., cells diseased with the type of disease treated by the therapeutic agent). Thus, in a preferred embodiment, a standard profile can be utilized to generate a standard signature.

Furthermore, the language “standard signature” includes information regarding the small molecules of the signature that is necessary and/or sufficient to provide information to a user for its intended use within the methods described herein. The standard signature would include the quantity and/or type of small molecules present. The ordinarily skilled artisan would know that the information which is necessary and/or sufficient will vary depending on the intended use of the “standard signature.” For example, the “standard signature,” can be determined using a single technique for an intended use but may require the use of several different techniques for another intended use depending on such factors as the types of small molecules present in a particular targeted cellular compartment, the cellular compartment being assayed per se. etc.

The relevant information in a “standard signature” also may vary depending on the intended use of the compiled information, e.g. spectra. For example for some intended uses, the amounts of a particular small molecule or a particular class of small molecules of the standard signature may be relevant, but for other uses the distribution of types of small molecules of the standard signature may be relevant.

Furthermore, comparison of the standard profiles and signatures to profiles and signatures from diseased cells can be used to identify small molecules deregulated in the disease state. The small molecules identified can be used to guide the drug discovery effort. For example, the small molecules present in aberrant levels in the sample cells can be identified and used as pharmaceutical agents. For example, if a patient is suffering from a disease state associated with a aberrantly low level of a certain compound, the compound or a precursor thereof may be tested in an assay that mimics the disease state. In another embodiment, the small molecules present in aberrant amounts may be used as targets for drug design to develop agents with enhanced activity, e.g., enhanced activity to treat the disease state associated with the aberrant levels of the small molecule. Additionally libraries of small molecules based on the structures of the small molecules present in aberrant amounts can be used to develop more potent therapeutics. The cellular targets and pathways could also be used to guide drug design.

In a further embodiment, the invention pertains to a method for treating a patient with a deficiency in certain disease relevant small molecules. The method includes obtaining cells from the patient, obtaining the small molecule profile of either a particular organelle (e.g., mitochondria, nucleus, cytoplasm, Golgi apparatus, endoplasmic reticulum, etc.) or a cell, generating a small molecule signature from many small molecule profiles, comparing the small molecule signature with a standard signature, determining a deficiency in the patient's small molecule signature of a certain disease relevant small molecule, and administering the disease relevant small molecule to the patient.

In a further embodiment, the invention features diagnostic assays for the detection of disease states. For example, the method includes identifying one or more small molecules which are present in aberrant amounts in a particular disease state, e.g., by comparing profiles of cells, fluids or cellular compartments from diseased patients with those from healthy patients to identify compounds which are present in aberrant amounts in the diseased patient. The method also involves designing a reagent that specifically reacts with the compound or compounds present in aberrant amounts to indicate the presence or absence of the compound or compounds, and therefore, the presence or the absence of the disease. The invention also pertains to kits which include the reagent and instructions for its use to diagnose the disease.

In a further embodiment, the invention features a method for monitoring the disease progression of a patient during the course of a treatment, for the purposes of determining the effectiveness of the chosen treatment. For example, the method includes diagnosing a disease in a patient as described above. During the course of a subsequent treatment, multiple small molecule profiles can be generated from samples from taken from the patient at varying times during the treatment. If the similarity of the profile to the small molecule disease signature lessens over the course of the treatment, then the treatment is being effective. Conversely, little change in the small molecule profile during the course of the treatment can mean that the treatment is ineffective for said patient.

Methods of Identifying the Effect of Chemical Agents on Small Molecule Signatures of Cells, Cellular Compartments and Organelles

In another aspect, the invention pertains to the comparison of small molecule profiles of cells, cellular compartments, or organelles with those of cells, cellular compartments, or organelles treated with toxins, chemical agents or therapeutic agent (or derived from an organism treated with the agent or drug). A therapeutic agent is also referred to as a drug. In preferred embodiments, the signature generated from drug, chemical or therapeutic agent treatment is an “efficacy signature” and a signature generated from toxicant treatment is a “toxicity signature.” In one embodiment, the cells, cellular compartments, or organelles are diseased (or derived from a diseased organism) and are treated with a therapeutic agent which is known to modify or treat that disease. For example, the small molecule signature of a cell treated with a therapeutic agent, chemical agent, or toxin, can be compared the small molecule signature of a normal cell, e.g., a healthy cell of similar lineage, or a diseased cell of similar lineage which was not treated with the therapeutic agent, chemical agent, or toxin. Examples of toxins include bacterial toxins such as endotoxins and exotoxins, such as cholera toxin, diptheria toxin, verotoxin, enterotoxin, etc. In a further embodiment, the cells are genetically altered.

In one embodiment, the biological samples used for drug efficacy or toxicity experiments can be treated with a specific drug or toxicant and compared to those treated with a placebo. Varying doses can be administered, and samples can be taken at varying times after dosing.

In addition, subtraction signatures can be obtained by subtracting the nontreated signature or a standard signature with the small molecule signature generated from many small molecule profiles from a treated cell, cellular compartment, or organelle. The subtraction signatures can then be used to identify certain small molecules the presence or the absence of which may indicate the efficacy or the toxicity of the compound. The subtraction signatures can be made using, for example, computer programs known to those of skill in the art. An example of such computer programs is disclosed below. It should be noted that the comparison of the signatures can be done both quantitatively and qualitatively.

In a further embodiment, the invention pertains to certain small molecules which indicate the efficacy or the toxicity of the compound. The invention also applies to assays which can be developed to indicate the presence or absence of these certain small molecules. For example, if the presence of a certain small molecule is essential for the efficacy of a particular therapeutic compound, then an assay can be developed to quickly determine the presence or absence of this certain small molecule in cell samples treated with test compounds. This can be both an effective and inexpensive method to determine the potential efficacy of compounds. It can be used alone or in combination with traditional drug screening assays such as, for example, binding assays and other enzymatic assays.

For example, in search of molecules with anti-tumor activity, small molecule signatures could be generated from small molecule profiles taken of cells at certain intervals after being treated with a known anti-tumor drug (e.g., taxol, cisplatin, adriamycin, etc.). Comparison of the small molecule signatures of these cells could lead to the identification of small molecules regulated by these drug. The identified small molecules could then be used to guide drug discovery by pointing to pathways which could be targeted for drug design or by using them as therapeutic or nutriceutical agents. Furthermore, both the targets and the identified small molecules can be used in assays of the invention described in detail in later sections.

The invention also includes a method for determining the toxicity of a test compound, e.g., a compound in development as a therapeutic agent. The method includes culturing cells, contacting a portion of the cells with the test compound, taking small molecule profiles of the cells contacted with the test compound to generate a small molecule signature, taking small molecule profiles of cells not contacted with the test compound to generate an untreated small molecule signature, and comparing the signatures to either each other or signatures from cells contacted with a known therapeutic agent or cells contacted with a known toxin. The method also can include a step of purifying a particular organelle of interest from the cells and obtaining the small molecule signature of the particular organelle of interest (e.g., nuclei, mitochondria, Golgi apparatus, endoplasmic reticulum, ribosome, etc.).

In a further embodiment, the invention pertains to a method for reducing side effects of drugs under development. For example, cells can be cultured, contacted with the test compound, the small molecule signature can be generated from many small molecule profiles, and compared to the signatures of known toxins and therapeutic agents. Changes then can be made to the structure of the test compound to reduce the side effects. For example, in order to test for liver toxicity, the compound may be incubated the in a liver cell culture to mimic the biotransformation that occurs in the liver. The small molecule signatures of cells and organelles in the treated and untreated liver cultures can be compared to the small molecule signatures of known toxins. Both the total cellular small molecule signature could be compared or the small molecule signature of a particular organelle, e.g., mitochondria, Golgi apparatus, nuclei, ribosomes, endoplasmic reticulum, etc.

The methods of the invention are particularly useful because they offer a quick and relatively inexpensive method to determine whether a certain test compound is likely toxic to a body organ, such as the liver. This allows pharmaceutical companies to quickly screen and identify compounds which are toxic and to direct their research towards non-toxic compounds.

The methods and small molecule signatures of the invention may also be used to rescue drugs, e.g., drugs which fail a particular step in the clinical or pre-clinical trial procedure. The failed drug can be exposed to cells or a test organism and small molecule profiles of the cells, cellular compartments, organelles, etc. can be taken and used to generate a small molecule signature, which can be compared to those of known toxins, known therapeutic agents, etc. to pinpoint the reason for failure of the drug. Small molecule signatures of various organs can also be taken if it is advantageous for the study (e.g., small molecule signatures can be generated from many small molecule profiles taken from muscle, brain, retinal, nerve, heart, lung, stomach, colon, skin, breast, fatty tissue, blood, etc.) Then the drug can be redesigned to avoid the its previous adverse effects.

The methods and small molecule signatures of the invention can also be used to “reposition” drugs. The term “reposition” refers to discovering new uses for an agent. In one embodiment, a dose of an agent is administered to a subject (e.g., a human or other animal, healthy or diseased) and small molecule signatures are generated from many small molecule profiles taken from various organs, tissues, cells, cellular compartments, and/or organelles of the subject to determine what tissues, organs, cells, cellular compartments, and/or organelles are being affected by the administration of the agent.

Methods of Identifying Small Molecules Associated with Body Weight Disorders

The invention also pertains to methods for identifying small molecules associated with, for example, body weight disorders such as obesity. Examples of methods for identifying small molecules associated with body weight disorders are described below. The following experiments are directed to the identification of small molecules associated with short-term appetite control. These experiments can be used to identify small molecules involved in signaling hunger and satiety.

In one embodiment, test subjects, preferably mice, will be fed normally prior to the initiation of the experiment, and then divided into one control and two experimental groups. The control group will then be maintained on ad lib nourishment, while the first experimental group (“fasted group”) will be fasted, and the second experimental group (“fasted-refed group”) will initially be fasted, and will then be offered a highly palatable meal shortly before the collection of tissue samples. Each test animal will be weighed immediately prior to and immediately after the experiment. Small molecule profiles will be taken of each mouse from each group and used to generate a small molecule signature. The signatures of each group will be averaged and compared to those of different groups.

Other experiments which may be used for the identification of cellular small molecules involved in, for example, body weight disorders, are experiments designed to analyze small molecules which may be involved genetic obesity. In the case of mice, for example, such experiments may identify small molecules regulated by the ob, db, and/or tub gene products. In the case of rats, for example, such paradigms may identify small molecules regulated by the fatty (fa) gene product.

In one embodiment of such an experiment, test subjects may include ob/ob, db/db, and/or tub/tub experimental mice and lean littermate control animals. The animals would be offered normal nourishment for a given period, after which tissue samples would be collected for analysis.

In additional experiments, ob/ob, db/db, and/or tub/tub experimental mice and lean control animals may be used as part of the short term appetite control experiments discussed above, or in other experiments discussed herein, such as set-point experiments and drug related experiments.

Experiments which may be used for the identification of small molecules involved in body weight disorders may include experiments designed to identify those small molecules which may be regulated in response to changes in body weight, e.g., “set point experiments”.

In one experiment, test subjects, preferably mice, will be fed normally prior to the initiation of the experiment, and then divided into one control and two experimental groups. The control group will then be maintained on an ad lib diet of normal nourishment in order to calculate daily food intake. The first experimental group (“underweight group”) will then be underfed by receiving some fraction of normal food intake, 60-90% of normal, for example, to reduce and maintain the group's body weight to some percentage, for example 80%, of the control group. The second experimental group (“overweight group”) will be overfed by receiving a diet which would bring the group to some level above that of the control, for example 125% of the control group. Tissue samples will then be obtained for analysis to identify small molecules which are present in different amounts in control versus overweight and/or underweight conditions.

Additionally, human subjects may be used for the identification of obesity-associated small molecules. In one embodiment of such an experiment, tissue samples may be obtained from obese and lean human subjects and analyzed for the presence of small molecules which are present in different amounts in the tissue, cells, or cellular organelles of one group as opposed to another (e.g. differentially present in lean versus obese subjects). In another embodiment, obese human subjects may be studied over the course of a period of weight loss, achieved through food restriction. Tissue from these previously obese subjects may be analyzed for differing amounts of small molecules relative to tissue obtained from control (lean, non-previously obese) and obese subjects.

Experiments may also be designed to identify small molecules involved in body weight disorders and may also include experiments designed to identify small molecules associated with body weight disorders induced by some physical manipulation to the test subject, such as, for example, hypothalamic lesion-induced body weight disorders. For example, bilateral lesions in the ventromedial hypothalamus (VMH) of rodents may be utilized to induce hyperphagia and gross obesity in test subjects, while bilateral lesions in the ventrolateral hypothalamus (VLH) of rodents may be used to induce aphagia in test subjects. In such experiments, tissue from hypothalamic-lesioned test subjects and from control subjects would be analyzed for the identification of small molecules which are present in different amounts in control versus lesioned animals.

Drugs known to affect (e.g., ameliorate) human or animal body weight and/or appetite (such as short term appetite) may be incorporated into the experiments designed to identify small molecules which are involved in body weight disorders and/or body weight or appetite regulation. These compounds may include known therapeutics, as well as compounds that are not useful as therapeutics due to, for example, their harmful side effects. Among the categories of control and test subjects which may be used in such experiments are, for example, lean subjects, obese subjects, and obese subjects which have received the drug of interest. In variations of the experiment, subjects such as these may be fed a normal ad lib diet, a caloric restriction maintained diet, or a caloric restriction ad lib diet. Control and test subjects may additionally be pairfed i.e., the control and test subjects may be fed via a coupled feeding device such that both control and test subjects receive identical amounts and types of food).

Methods of Identifying Small Molecules Associated with Immunological Diseases

The invention also pertains to methods for identifying small molecules associated with, for example, normal and abnormal immune responses. Examples of methods for identifying small molecules associated with immune responses are described below. The following experiments are directed to the identification of small molecules which are differentially present within and among TH cell subpopulations, including but not limited to TH1 and TH2 subpopulations. Such small molecules can be involved in, for example, TH cell subpopulation differentiation, maintenance, and/or effector function, and in TH cell subpopulation-related disorders. For example, TH cells can be induced to differentiate into either TH1 or TH2 states, can be stimulated with, for example, a foreign antigen, and can be collected at various points during the procedure for analysis of their small molecule signatures generated from small molecule profiles. This example is merely meant to be illustrate several experiments which can be done using small molecule signatures generated from small molecule profiles to determine small molecules associated with immunological disorders. This example is not intended to limit the invention to the specific types of cells or subjects discussed in this section.

In one experiment, transgenic animals, preferably mice, will be used which have been engineered to express a particular T cell receptor, such that the predominant T cell population of the immune system of such a transgenic animal recognizes only one antigen. Such a system will be used because it provides a source for a large population of identical T cells whose naivete can be assured, and because its response to the single antigen it recognizes is also assured. T helper cells can be isolated from such a transgenic animal can then be induced, in vitro, to differentiate into TH cell subpopulations such as TH1, TH2, or TH0 cell subpopulations. In one embodiment, one T helper cell group (the TH1 group) is exposed to IL-12, a cytokine known to induce differentiation into the TH1 state, a second T helper cell group (the TH2 group) is exposed to IL-4, a cytokine known to induce differentiation into the TH2 state, and a third group is allowed, by a lack of cytokine-mediated induction, to enter a TH-undirected state. Small molecule profiles of each type of cells can then be taken, used to generate small molecule signatures and compared.

In another experiment, mature TH cell clones can be used, such as TH1 and TH2 and TH1-like and TH2-like cell lines, preferably human cell lines. Such TH cell lines can include, but are not limited to the following well known murine cell lines: Doris, AE7, D10.G4, DAX, D1.1 and CDC25. Such T cell lines can be derived from normal individuals as well as individuals exhibiting TH cell subpopulation-related disorders, such as, for example, chronic inflammatory diseases and disorders, such as Crohn's disease, reactive arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease, sarcoidosis, atopic conditions, such as asthma and allergy, including allergic rhinitis, gastrointestinal allergies, including food allergies, eosinophilia, conjunctivitis, glomerular nephritis, certain pathogen susceptibilities such as helminthic (e.g., leishmaniasis) and certain viral infections, including HIV, and bacterial infections, including tuberculosis and lepromatous leprosy.

The TH cell clones can be stimulated in a variety of ways. Such stimulation methods include, but are not limited to, pharmacological methods, such as exposure to phorbol esters, calcium ionophores, or lectins (e.g., Concanavalin A), by treatment with antibodies directed against T-cell receptor epitopes (e.g., anti-CD3 antibodies) or exposure, in the presence of an appropriate antigen presenting cell (APC), to an antigen that the particular TH cells are known to recognize. Following such primary stimulation, the cells can be maintained in culture without stimulation and, for example, in the presence of IL-2, utilizing standard techniques well known to those of skill in the art. The cells can then be exposed to one or more additional cycles of stimulation and maintenance. The small molecule profiles of the cells and cellular compartments can be taken at any time during the process of the stimulation in this experiment and used to generate small molecule signatures.

A third experiment can also be used to discover small molecules present in different amounts. In vivo stimulation of animal models forms the basis for this experiment. The in vivo nature of the stimulation can prove to be especially predictive of the analogous responses in living patients. Stimulation can be accomplished via a variety of methods. For example, animals, such as transgenic animals described earlier, can be injected with appropriate antigen and appropriate cytokine to drive the desired TH cell differentiation. Draining lymph nodes can then be harvested at various time points after stimulation. Lymph nodes from, for example, TH1-directed animals can be compared to those of TH2-directed animals. A wide range of animal models, representing both models of normal immune differentiation and function as well as those representing immune disorders can be utilized for this in vivo experiment.

Cell or organelle samples can be collected during any point of such a procedure for small molecule profiling and signature production. For example, cells or organelles can be obtained following any stimulation period and/or any maintenance period. Additionally, the cells or organelles can be collected during various points during the TH cell differentiation process. The small molecule profiles can be taken to generate signatures of the cells or organelles, which can be compared using the methods outlined herein. For example, small molecule signatures generated from small molecule profiles of TH0, TH1 and TH2 groups isolated at a given time point can then be analyzed and compared. Additionally, small molecule signatures generated from small molecule profiles of stimulated and non-stimulated cells within a given TH cell group can also be compared and analyzed. Further, small molecule signatures from undifferentiated TH cells can be compared to small molecule signatures from cells at various stages during the differentiative process which ultimately yields TH cell subpopulations.

Methods of Identifying Small Molecules Associated with Cardiovascular Disorders

The small molecule signatures of the invention can be used to identify small molecules which are relevant to cardiovascular disease. According to the invention, signatures are generated from small molecule profiles for small molecules present in endothelial cells or endothelial cell organelles subject to fluid shear stress in vitro. Shear stress may be responsible for the prevalence of atherosclerotic lesions in areas of unusual circulatory flow.

Cell cultures are exposed to fluid shear stress which is thought to be responsible for the prevalence of atherosclerotic lesions in areas of unusual circulatory flow. Unusual blood flow also plays a role in the harmful effects of ischemia/reperfusion, wherein an organ receiving inadequate blood supply is suddenly reperfused with an overabundance of blood when the obstruction is overcome.

Cultured HUVEC monolayers are exposed to laminar shear stress by rotating the culture in a specialized apparatus containing liquid culture medium (Nagel et al., 1994, J. Clin. Invest. 94: 885-891). Static cultures grown in the same medium serve as controls. After a certain period of exposure to shear stress, experimental and control cells will be harvested, organelles isolated and small molecule signatures will be generated from the small molecule profiles to identify molecules which are present in exposed versus control cells.

In experiments designed to identify small molecules which are involved in cardiovascular disease, compounds such as drugs known to have an ameliorative effect on the disease symptoms may be incorporated into the experimental system. Such compounds may include known therapeutics, as well as compounds that are not useful as therapeutics due to their harmful side effects. Test cells that are cultured, for example, may be exposed to one of these compounds and analyzed for different small molecule signatures generated from small molecule profiles with respect to untreated cells, according to the methods described herein. In principle, according to the particular experiment, any cell type involved in the disease may be treated at any stage of the disease process by these therapeutic compounds.

Test cells may also be compared to unrelated cells (e.g., fibroblasts) that are also treated with the compound, in order to screen out generic effects on small molecule signatures that may not be related to the disease. Such generic effects might be manifest by changes in small molecule signatures that are common to the test cells and the unrelated cells upon treatment with the compound.

By these methods, the small molecules upon which these compounds affect can be identified and used in the assays described below to identify novel therapeutic compounds for the treatment of cardiovascular disease.

In another experiment, small molecules are identified from monocytes from human subjects. This experiment involves differential treatment of human subjects through the dietary control of lipid consumption. The human subjects are held on a low fat/low cholesterol diet for three weeks, at which time blood is drawn, monocytes are isolated according to the methods routinely practiced in the art, organelles, such as mitochondria, nuclei, and the cytosol, are isolated and signatures are generated. These same patients are subsequently switched to a high fat/high cholesterol diet and monocyte organelles are purified again. The patients may also be fed a third, combination diet containing high fat/low cholesterol and monocyte organelles may be purified once again. The order in which patients receive the diets may be varied. The small molecule profiles of the organelles derived from patients maintained on two of the diets, or on all three diets, can be used to generate small molecule signatures, which may then be compared and analyzed.

In addition to the detection of different small molecule profiles and the subsequent generation of small molecule signatures in monocytes, paradigms focusing on endothelial cells may be used to detect small molecules involved in cardiovascular disease. In one experiment, human umbilical vein endothelial cells (HUVEC's) are grown in vitro. Experimental cultures will then be treated with human IL-1β, a factor known to be involved in the inflammatory response, in order to mimic the physiologic conditions involved in the atherosclerotic state. Alternatively experimental HUVEC cultures may be treated with lysophosphatidylcholine, a major phospholipid component of atherogenic lipoproteins or oxidized human LDL. Control cultures are grown in the absence of these compounds. After a certain period of treatment, experimental and control cells will be harvested and small molecule profiles will be taken of the cells and/or organelles, which will be used to generate small molecule signatures to analyze.

Methods of Identifying Small Molecules Associated with Central Nervous System and Other Neurological and Neurodegenerative Disorders

The small molecule signatures of the invention can be used to identify small molecules which are relevant to central nervous system and other neurological and neurodegenerative disorders. Examples of such disorders include, for example, neuropathies, Alzheimer disease, Parkinson's disease, Huntington's disease, amyotropic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, peripheral nervous system diseases and mental disorders such as depression and schizophrenia, etc.

One method for identifying small molecules which are relevant to central nervous system and other neurological and neurodegenerative disorders, is to compare the small molecule signatures generated from small molecule profiles of a diseased cell, cellular compartment or organelle of a diseased organism to a small molecule signature generated from small molecule profiles of a healthy cell, cellular compartment, or organelle (e.g., a standard small molecule signature) For example, the cells can be derived from the subjects' brain, muscle, retinal, nerve tissue, spinal fluid, blood, etc.

The diseased organism can be either a human or animal patient suffering from a neurological disorder or from an animal model of such a disorder. In certain embodiments, the invention pertains to the small molecules which are found in aberrant amounts in the small molecule signatures of diseased cells. In other embodiments, the invention pertains to the small molecule subtraction signatures of particular neurological disorders (e.g., subtraction signatures of the diseased small molecule signature compared to the standard small molecule signature, etc.).

Methods of Identifying Small Molecules Associated with Oncological Disorders

In one embodiment, the invention pertains to methods of identifying small molecules associated with oncological disorders, e.g., cancerous tumors, leukemia, lymphoma, etc. In another embodiment, small molecules associated with an oncological disorder are identified by comparing small molecule signatures generated from small molecule profiles of cancerous tissue with normal tissue. In a further embodiment, the tissue is from the same individual, e.g., normal and malignant prostate tissues are excised from a mammalian subject, e.g., mouse, rat, or human. Small molecule signatures generated from small molecule profiles of cells, cellular compartments, or organelles of the normal tissue is compared with the corresponding small molecule signatures of the malignant tissue. When the small molecule signatures are compared, certain small molecules may appear to be present in aberrant amounts in the cancerous tissue.

The invention also pertains to methods for detecting aberrant amounts of the identified compound in other tissue, e.g., the methods of the invention can be used to develop a reagent that specifically reacts with cancerous tissue.

Methods of Identifying Small Molecules Regulated by Genes of Interest

In another embodiment, the invention pertains to methods of identifying small molecules regulated, modulated, or associated with genetic modification or alterations of cells, both engineered and naturally occurring. The identified small molecules can be used, for example, to determine the function of unknown genes in functional genomics. For example, the comparison of the small molecules found in genetically altered cells can be used to elucidate the function of any given gene. For example, the invention pertains to a method for identifying small molecules associated with expression vectors of interest by comparing the small molecules of host cells expressing an expression vector to the small molecules of host cells not expressing the expression vector. In one embodiment, the expression vector comprises a portion or fragment of the genome, e.g., human genome. In another embodiment, the expression vector may be known to be associated with a particular disease state. The small molecules of the cells with and with out the expression vector expressed can be used to identify small molecules of interest, pathways of interest, and targets for drug design and/or future study.

In a further embodiment, the small molecules of the cells are identified by through separation techniques such as mass spectroscopy, HPLC, and coulometric array technology to create small molecule signatures (see, for example, Kristal, B. S. et al. Anal. Biochem. 263:18-25 (1998)). The resulting small molecule signature can then be compared to the small molecule signature of other cells, e.g., cells not genetically modified.

The term “vector” includes nucleic acid molecules capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc.

The recombinant expression vectors of the invention can be designed for expression in prokaryotic or, preferably, eukaryotic host cells. For example, the vectors can be expressed in bacterial cells such as E. coli, insect cells (using baculovirus expression vectors) yeast cells or mammalian cells. In a preferred embodiment, mammalian cells include human cells. Suitable host cells are discussed further in Goeddel, Gene Expression Technology: Methods in Enzymology 185, Academic Press, San Diego, Calif. (1990).

Expression of vectors in prokaryotes is most often carried out in E. coli with vectors containing constitutive or inducible promoters directing the expression of either fusion or non-fusion proteins. Examples of inducible non-fusion E. coli expression vectors include pTrc (Amann et al., (1988) Gene 69:301-3.15) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident prophage harboring a T7 gn1 gene under the transcriptional control of the lacUV 5 promoter.

In another embodiment, the expression vector is a yeast expression vector. Examples of vectors for expression in yeast S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, the vector can be expressed in insect cells using baculovirus expression vectors. Baculovirus vectors available for expression of vectors in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In a preferred embodiment, a nucleic acid of the interest is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

The terms “host cell” and “recombinant host cell” are used interchangeably. These cells include not only the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

For this method, a host cell can be any prokaryotic or eukaryotic cell. For example, a protein of interest can be expressed in bacterial cells such as E. coli, insect cells, yeast or, preferably, mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. The terms “transformation” and “transfection” include a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G4 18, hygromycin and methotrexate. Nucleic acid encoding a selectable marker can be introduced into a host cell on the same vector as the gene or a separate vector. Cells stably transfected with the introduced nucleic acid can be identified by drug selection (e.g., cells that have incorporated the selectable marker gene will survive, while the other cells die).

Furthermore, in yet another embodiment, the invention also pertains to methods for identifying small molecules regulated by a gene expressed in a particular host cell. In this embodiment, the gene is removed, functionally disrupted, otherwise not expressed in the cell and the small molecules of the cell are compared to those of a similar cell wherein the gene is expressed. The small molecules which are regulated, modulated or associated with this gene can then be identified by the comparison of the small molecule signature generated from the small molecule profiles of the cells with and without the gene expressed. The small molecules which are present in aberrant amounts can then be used to identify pathways, targets, and other small molecules associated with this gene, using methods of the invention.

To functionally disrupt a gene of a cell, a vector is prepared which contains at least a portion of a gene of interest into which a deletion, addition or substitution has been introduced to thereby alter, e.g., functionally disrupt, the gene of interest. The gene of interest can be a human gene, or a non-human homologue of a human gene. In an embodiment, the vector is designed such that, upon homologous recombination, the endogenous gene of interest is functionally disrupted (i.e., no longer encodes a functional protein; also referred to as a “knock out” vector). Alternatively, the vector can be designed such that, upon homologous recombination, the endogenous gene of interest is mutated or otherwise altered but still encodes, for example, a functional protein (e.g., the upstream regulatory region can be altered to thereby alter the expression of the endogenous protein). In the homologous recombination vector, the altered portion of the gene of interest is flanked at its 5′ and 3′ ends by additional nucleic acid sequence of the gene of interest to allow for homologous recombination to occur between the exogenous gene of interest carried by the vector and an endogenous gene of interest in a cell. The additional flanking nucleic acid sequence should be of sufficient length for successful homologous recombination with the endogenous gene. Typically, several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into a cell line (e.g., by electroporation) and cells in which the introduced gene of interest has homologously recombined with the endogenous gene of interest are selected (see e.g., Li, E. et al. (1992) Cell 69:915). The small molecule signature of the gene disrupted cells can then be compared to the small molecule signature of the cells without the gene of interest disrupted, thus identifying small molecules associated with the gene of interest.

Assays for Identifying Potential Cell Drug Targets Using Labeled Disease Relevant Small Molecules

In another embodiment, the invention also pertains to methods for identifying potential cell drug targets (e.g., cellular components which interact with the labeled small molecules). This method is particularly useful because it can identify components which are known to interact with disease relevant small molecules. Therefore, targets identified through this method are “pre-validated,” and some of the guess work surrounding the choice of target is eliminated. In a further embodiment, this method can be used in conjunction with conventional genomics as a further validation step to identify targets for further research.

The method includes obtaining a cell from a source, obtaining samples of small molecules from the cell; testing the samples for biological activity; identifying the biologically active small molecules of the samples; labeling the biologically active small molecules; contacting the labeled small molecules with cellular components; and identifying interactions between cellular components and said labeled small molecules. The invention includes the identified cell drug targets as well as the identified biologically active small molecules.

In another embodiment, the invention includes a method for identifying potential cell drug targets. The method includes contacting a labeled disease relevant small molecule with cellular components; and identifying interactions between said cell components and the labeled disease-relevant small molecule.

The labeled small molecules also include labeled “disease-relevant small molecules,” identified by any of the techniques described herein (e.g., comparison of small molecule signatures in healthy and diseased cells, etc.). In another embodiment, the method includes contacting a labeled disease relevant small molecule with cellular components, and identifying the interactions between the cellular-components and the labeled disease relevant small molecule.

The term “label” includes any moieties or molecules which enhance the ability of the labeled small molecules to be detected. Examples of suitable labels are well known in the art. radiolabels and fluorescent labels. The term “label” includes direct labeling of the small molecule by radiolabeling, coupling (i.e., physically linking) a detectable substance (e.g., a fluorescent moiety) to the small molecule, and indirect labeling of the small molecule by reacting the small molecule with another reagent that is directly labeled. Examples of indirect labeling include detection of a small molecules by labeling it with biotin such that it can be detected with fluorescently labeled streptavidin. In one embodiment, the small molecules are fluorescently labeled or radiolabeled.

The term “cellular components” includes material derived from cells. The cellular components can be purified or crude cellular extracts. The cellular components can be derived from one type of cell, or even a specific cellular compartment such as an organelle (e.g., mitochondria, nucleus, cytoplasm). Furthermore, the term includes both natural proteins found within biological systems and chimeric and other engineered proteins. In one embodiment, the term “cellular component” includes cellular receptors. The term also includes natural and unnatural polysaccharides and nucleic acids. In one embodiment, the term “cellular component” is a crude cellular extract from a human cell. The term “cellular component” includes “targets.”

Samples of the invention that bind to cellular components can be identified by preparing a reaction mixture of the cellular components and the samples under conditions and for a time sufficient to allow the components and the sample to interact and bind, thus forming a complex which can be removed and/or detected in the reaction mixture. The cellular components used can vary depending upon the goal of the screening assay. In one embodiment, the sample of the invention is an isolated, labeled small molecule, e.g., a disease relevant small molecule, a small molecule with biological activity or another small molecules which is present in aberrant levels in disease states. The assay can be used to determine which cellular components the small molecule interacts with. The identified cellular components which interact with the small molecule can then be used for drug design.

In a further embodiment, the cellular components are a nucleic acid array. High density arrays of nucleic acids (such as cDNA's and synthetic oligonucleotides) allow for a high degree of automation, repetitive analysis and duplication at minimal cost (Fraser, Electrophoresis, 18:1207-1215 (1997)). The development of recent technology has provided methods for making very large arrays of oligonucleotide probes in very small areas (see, for example, U.S. Pat. No. 5,143,854, WO 90/15070 and WO 92/10092, each of which is incorporated herein by reference). In one embodiment, the nucleic acids of the array are human genes. Examples of nucleic acid arrays include those mentioned in U.S. Pat. No. 6,027,880 and U.S. Pat. No. 5,861,242. The nucleic acids also can be representative of RNA molecules present in a cell, tissue or organ (e.g., the “transcriptome”, see Hoheisel, J. et al. Trends Biotechnol. 15:465-469 (1997); Velculescu, Cell, 88:243-251 (1997)). In one embodiment, the nucleic acids are in array.

In another further embodiment, the cellular components are a protein array. Examples of protein arrays include those employing conventional protein separation techniques, such as 2-dimensional gel electrophoresis, chromatographic procedures (e.g., FPLC, SMART by Pharmacia, Uppsala, Sweden), capillary electrophoretic techniques and mass spectrometry. In another embodiment, the protein array is a soup of proteins that contains a significant portion of the diversity encoded by a genome (see WO 99/39210).

In a further embodiment, the cellular components are a 2D protein gel. The 2D protein gel may be a complete or an incomplete set of the protein molecules present in a cell, tissue or organ (e.g., the proteome, see Sagliocco, et al. Yeast 12, 15191534 (1996); Shevalanko, et al. Porch. Nat. Acad. Sci. 93, 14440-14445 (1996)). Labeled biologically active small molecules previously identified through methods of the invention can then be contacted with the 2D gels and interactions between the labeled small molecules and the protein of the 2D gel can be detected.

The proteins identified through this method can then be further tested for biological activity, e.g., biological activity relating to that of the small molecule, e.g., through knock-out mice, inhibition studies, and other techniques known in the art. Furthermore, the identified proteins can then be used in drug design to identify other molecules (either naturally occurring or chemically synthesized) which bind or interact with the protein which may have advantageous characteristics (e.g., enhanced biological activity, less toxic side effects).

Predictive Medicine and Pharmacometabolomics

The present invention also pertains to the field of predictive medicine in which diagnostic assays, prognostic assays, pharmacometabolomics, and monitoring clinical trials are used for prognostic (predictive) purposes to thereby treat an individual prophylactically. Accordingly, one aspect of the present invention relates to diagnostic assays for determining small molecule signatures, in the context of a biological sample (e.g., blood, serum, cells, tissue, cellular organelles) to thereby determine whether an individual is afflicted with a disease or disorder, or is at risk of developing a disorder, associated with aberrant levels of small molecules. The invention also provides for prognostic (or predictive) assays for determining whether an individual is at risk of developing a disorder associated with relevant small molecules. For example, aberrant levels of small molecules can be signatured from a small molecule profile of a biological sample. Such assays can be used for prognostic or predictive purpose to thereby prophylactically treat an individual prior to the onset of a disorder characterized by or associated with a relevant small molecule.

Another aspect of the invention provides methods for determining small molecule signatures generated from small molecule profiles of an individual to thereby select appropriate therapeutic or prophylactic agents for that individual (referred to herein as “pharmacometabolomics”). Pharmacometabolomics allows for the selection of agents (e.g., drugs) for therapeutic or prophylactic treatment of an individual based on the small molecule signature of the individual (i.e., the individual's “metaboprint”). The metaboprint of the individual is examined to predict what the person's reaction to a particular therapeutic compound will be. Yet another aspect of the invention pertains to monitoring the influence of agents (e.g., drugs or other compounds) on the small molecule signatures of the patients in clinical trials.

Pharmacometabolomics is similar to pharmacogenomics but it is also able to taken in to account environmental and other non-genetic factors (e.g., other drugs, etc.) which may affect an individual's response to a particular therapeutic compound. Pharmacometabolomics can be used alone or in combination with pharmacogenomics to predict an individual's reaction to a particular drug based upon their metaboprint (e.g., small molecule signature) and/or their genotype.

Pharmacometabolomics is particularly useful because it provides an early warning sign, due to its capability of detecting aberrant small molecules long before any disease symptoms or predisposed phenotypes are noticed.

Diagnostic Assays: In one embodiment, the invention pertains to a method for facilitating the diagnosis of a disease state of a subject. The method includes obtaining a small molecule signature generated from small molecule profiles from a subject suspected of having and/or having a disease state, and comparing the small molecule signature from the subject to a standard small molecule signature.

The invention provides a method of assessing small molecule signatures, especially aberrant small molecule signatures. Aberrant small molecule signatures generated from small molecule profiles (e.g., excessive amounts of a particular molecule, deficient amounts of a particular molecule, the presence of a small molecule not usually present, etc.) may indicate the presence of a disease state. More generally, aberrant small molecule signatures may indicate the occurrence of a deleterious or disease-associated metaboprint contributed by small molecules present in aberrant amounts.

The standard small molecule signature can be generated from small molecule profiles obtained from healthy subjects or subjects afflicted with the disease state which is the subject is suspected of having. The small molecule signatures can be generated from small molecule profiles taken from a particular organ, tissue, or combinations or organs or tissues. The small molecule signatures can also be generated from small molecule profiles taken of cells, cellular compartments, or particular organelles.

The term “disease state” includes any states which are capable of being detected metabolomically by comparing small molecule signatures of a subject having the disease to a standard small molecule signature. Examples of disease states include, but are not limited to, include metabolic diseases (e.g., obesity, cachexia, diabetes, anorexia, etc.), cardiovascular diseases (e.g., atherosclerosis, ischemia/reperfusion, hypertension, restenosis, arterial inflammation, etc.), immunological disorders (e.g., chronic inflammatory diseases and disorders, such as Crohn's disease, reactive arthritis, including Lyme disease, insulin-dependent diabetes, organ-specific autoimmunity, including multiple sclerosis, Hashimoto's thyroiditis and Grave's disease, contact dermatitis, psoriasis, graft rejection, graft versus host disease, sarcoidosis, atopic conditions, such as asthma and allergy, including allergic rhinitis, gastrointestinal allergies, including food allergies, eosinophilia, conjunctivitis, glomerular nephritis, certain pathogen susceptibilities such as helminthic (e.g., leishmaniasis) and certain viral infections, including HIV, and bacterial infections, including tuberculosis and lepromatous leprosy, etc.), nervous system disorders (e.g., neuropathies, Alzheimer disease, Parkinson's disease, Huntington's disease, amyotropic lateral sclerosis, motor neuron disease, traumatic nerve injury, multiple sclerosis, acute disseminated encephalomyelitis, acute necrotizing hemorrhagic leukoencephalitis, dysmyelination disease, mitochondrial disease, migrainous disorder, bacterial infection, fungal infection, stroke, aging, dementia, peripheral nervous system diseases and mental disorders such as depression and schizophrenia, etc.), oncological disorders (e.g., leukemia, brain cancer, pancreatic cancer, prostate cancer, liver cancer, stomach cancer, colon cancer, throat cancer, breast cancer, ovarian cancer, skin cancer, melanoma, etc.). The term also include disorders which result from oxidative stress.

The term “subject” includes humans, animals, and plants. Preferably, the subject is human. In one embodiment, the subject is a human suffering from or at risk of suffering from a disease state.

The invention also encompasses kits for detecting the presence of a particular relevant small molecule in a biological sample (a test sample). Such kits can be used to determine if a subject is suffering from or is at increased risk of developing a disorder associated with the relevant small molecule (e.g, drug resistance). For example, the kit can comprise a labeled compound or agent capable of detecting the relevant small molecule in a biological sample and means for determining the amount of the relevant small molecule in the sample (e.g., an antibody against the relevant small molecule another molecular or chemical sensor). Kits may also include instruction for observing that the tested subject is suffering from or is at risk of developing a disorder associated with the relevant small molecule if the amount of the relevant small molecule is above or below a normal level.

The kit may also comprise, e.g., a buffering agent, a preservative, or a stabilizing agent. The kit may also comprise components necessary for detecting the detectable agent (e.g., a substrate). The kit may also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with the relevant small molecule.

Prognostic Assays: The invention also pertains to a method for predicting whether a subject is predisposed to having a disease state. The method includes a small molecule signature generated from small molecule profiles obtained from the subject; and comparing the small molecule signature from the subject to a standard small molecule signature, thereby predicting whether a subject is predisposed to having a disease state.

The methods described herein can furthermore be used as diagnostic or prognostic assays to identify subjects having or at risk of developing a disease or disorder associated with aberrant small molecule signatures. For example, the assays described herein, such as the preceding diagnostic assays or the following assays, can be utilized to identify a subject having or at risk of developing a disorder associated with an aberrant small molecule signature, such as drug resistance of tumor cells. Alternatively, the prognostic assays can be utilized to identify a subject having or at risk for developing such a disease or disorder. Thus, the present invention provides a method in which a test sample is obtained from a subject and a small molecule signature is generated from small molecule profiles taken from the test sample, wherein an aberrant small molecule signature is diagnostic for a subject having or at risk of developing a disease or disorder associated with an aberrant small molecule signature. The term “test sample” is a biological sample obtained from a subject of interest. For example, a test sample can be a biological fluid (e.g., serum), cell sample, or tissue. Advantageously, the test sample may consist of cells or individual organelles, e.g., mitochondria, nuclei, Golgi apparatus, endoplasmic reticulum, ribosomes, chloroplasts, etc.

Furthermore, the prognostic assays described herein can be used to determine whether a subject can be administered an agent (e.g., peptidomimetic, protein, peptide, nucleic acid, small molecule, or other drug candidate) to treat a disease or disorder associated with an aberrant small molecule signature. For example, such methods can be used to determine whether a subject can be effectively treated with a specific agent or class of agents (e.g., agents of a type which effect the small molecule signature in particular ways). Thus, the present invention provides methods for determining whether a subject can be effectively treated with an agent for a disorder associated with an aberrant small molecule signature in which a test sample is obtained and an aberrant small molecule signature generated from small molecule profiles is detected (e.g., wherein the presence or relative quantity of particular relevant small molecules is diagnostic for a subject that can be administered the agent to treat a disorder associated with the aberrant small molecule signature). In some embodiments, the foregoing methods provide information useful in prognostication, staging and management of particular states that are characterized by altered small molecule signatures and thus by a particular metaboprint. The information more specifically assists the clinician in designing treatment regimes to eradicate such particular states from the body of an afflicted subject.

The methods of the invention can also be used to detect the presence or absence of relevant small molecules, thereby determining if a subject is at risk for a disorder associated with this relevant small molecule. For example, the presence or absence of relevant small molecules, may indicate whether the process of developing a disease state has been initiated or is likely to arise in the tested cells. In preferred embodiments, the methods include detecting the presence or absence of the relevant small molecule, in a sample of cells from the subject, the presence or absence of a disease state. Preferably the sample of cells is obtained from a body tissue suspected of comprising diseased cells. Thus, the present method provides information relevant to diagnosis of the presence of a disease state. In one embodiment, the sample of cells is comprised mainly of a particular cellular organelle, e.g., mitochondria, Golgi apparatus, nuclei, etc.

The methods described herein may be performed, for example, by utilizing pre-packaged diagnostic kits comprising at least one reagent for detecting a relevant small molecule, which may be conveniently used, e.g., in clinical settings to diagnose patients exhibiting symptoms or family history of a disease or illness involving a relevant small molecule.

Pharmacometabolomics: The invention also pertains to a method for predicting a subject's response to a therapeutic agent. The method includes a small molecule signature generated from small molecule profiles obtained from the subject, and comparing the small molecule signature of the subject to a known standard established for the therapeutic agent as an indication of whether the subject would benefit from treatment with the therapeutic agent.

Agents, or modulators which alter levels of particular relevant small molecules, as identified by a screening assay described herein can be administered to individuals to treat (prophylactically or therapeutically) disorders associated with the relevant small molecules. In conjunction with such treatment, the pharmacometabolomics (i.e., the study of the relationship between an individual's metaboprint and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacometabolomics of the individual permits the selection of effective agents (e.g., drugs) for prophylactic or therapeutic treatments based on a consideration of the individual's metaboprint. Such pharmacometabolomics can further be used to determine appropriate dosages and therapeutic regimens. Accordingly, the small molecule signature generated from small molecule profiles of an individual can be utilized to select appropriate agent(s) for therapeutic or prophylactic treatment of the individual.

The known standard can be obtained from subjects who benefited from the agent, e.g., patients who were treated with the agent and were cured, maintained their health, or prevented or slowed the deterioration of health. The known standard can be taken from a particular tissue, organ. It can also be taken from any organelle, cell, or cellular compartment during any point during the beneficial treatment. It can be derived from a single patient or from an average of more than one patient who were treated successfully with the agent. In addition, the known standard can also be derived using other techniques.

Pharmacometabolomics deals with clinically significant hereditary and non-hereditary variations in the response to drugs due to altered drug disposition and abnormal action in affected persons. In general, several types of pharmacometabolomic conditions can be differentiated. For example, certain pharmacometabolomic conditions may be the result of genetic conditions. The genetic conditions may be transmitted as a single factor altering the way drugs act on the body (altered drug action) or genetic conditions transmitted as single factors altering the way the body acts on drugs (altered drug metabolism). These pharmacometabolomic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans. Examples of non-hereditary conditions which may affect the way drugs act on the body or the way the body acts on the drugs include the ingestion of certain drugs, the substance dependence of the patient, the diet of the patient, non-hereditary medical conditions of the patient, etc.

The small molecule signature and metaboprint of an individual can be utilized to select appropriate agent(s) for therapeutic or prophylactic treatment of the individual. In addition, pharmacometabolomic studies can be used to identify an individual's drug responsiveness metaboprint. This knowledge, when applied to dosing or drug selection, can avoid adverse reactions or therapeutic failure and thus enhance therapeutic or prophylactic efficiency when treating a subject with an agent, such as an agent identified by one of the exemplary screening assays known in the art.

Monitoring of Effects During Clinical Trials: The invention also pertains to a method for metabolomically monitoring the effectiveness of a therapeutic agent in clinical trials. The method includes a small molecule signature generated from small molecule profiles obtained from a subject in a clinical trial being treated with a therapeutic agent, and monitoring changes in the small molecule signature of the subject as an indication of the effectiveness of the therapeutic agent in the subject. In one embodiment, the small molecule signature of the subject can be compared to a predetermined standard.

Monitoring the influence of agents (e.g., drugs, therapeutic compounds) on the small molecule signature can be applied not only in basic drug screening, but also in clinical trials. For example, the effectiveness of an agent determined by a screening assay as described herein to increase levels of certain relevant small molecules, can be monitored in clinical trails of subjects exhibiting decreased levels of certain small molecules. Alternatively, the effectiveness of an agent determined by a screening assay to decrease levels of a certain relevant small molecule, can be monitored in clinical trails of subjects exhibiting increased levels of the certain relevant small molecule. In such clinical trials, the level of the certain small molecule and, preferably, the remainder of the small molecule signature can be used as a “read out” of the disease state of the particular cell.

For example, and not by way of limitation, small molecules that are modulated in cells by treatment with an agent (e.g., compound, drug or small molecule) can be identified in screening assays. The effect of agents on cellular proliferation disorders, for example, can be studied in a clinical trial. For example, cells can be isolated and small molecule signatures of either whole cells or particular organelles can be generated from small molecule profiles taken from the samples. In this way, the small molecule signature can serve as a marker, indicative of the physiological response of the cells to the agent. Accordingly, this response state may be determined before, and at various points during, treatment of the individual with the agent.

In another embodiment, the present invention provides a method for monitoring the effectiveness of treatment of a subject with an agent (e.g., peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the small molecule profiles of the pre-administration sample and generating a small molecule signature; (iii) obtaining one or more post-administration samples from the subject; (iv) detecting the small molecule profiles of the post-administration samples and generating a small molecule signature; (v) comparing the small molecule signature of the pre-administration sample with the small molecule signature of the post-administration sample or samples; and (vi) altering the administration of the agent to the subject accordingly. For example, increased administration of the agent may be desirable to increase the level of certain relevant small molecules to higher levels than detected, i.e., to increase the effectiveness of the agent. Alternatively, decreased administration of the agent may be desirable to decrease the level of certain relevant small molecules to lower levels than detected, i.e., to decrease the effectiveness of the agent.

Small Molecules Databases and Methods of Use

In one embodiment, the invention pertains to the creation of small molecule databases containing information regarding the metabolome of cells, cellular compartments, and organelles, e.g., cells, cellular compartments, and organelles in health, diseased, and altered states. The information regarding the small molecules of each cell, cellular compartment, or organelle can be found using the separation and analytical techniques described herein. The small molecule databases can include compounds derived from the same or different animal organs. For example, the small molecule databases can include compounds obtained from cells of specific organs such as a heart, brain, kidney, liver, done, blood, gastrointestinal tract, and/or muscle. In addition, the small molecule databases can include information regarding compounds obtained from individuals suffering from a particular disease state, e.g., cardiovascular diseases, neurodegenerative diseases, diabetes, obesity, immunological disorders, etc.

The databases can be made based on information obtained from the techniques described herein to determine the identity and presence of various small molecules in cells, cellular compartments, and organelles. The databases may include information regarding the compounds found, such as structure, molecular weight, amounts found in particular organelles in a particular state of health, and any other information that a person of skill in the art would consider relevant and useful to be contained in the database. For example, information regarding known biochemical pathways involving the particular compound may also be included as well as other such information.

In one embodiment, the databases of the invention contain information on the compounds of the metabolome of a particular organelle of a particular species in a particular state of health from a particular organ (e.g., one database may include compounds of the metabolome of the mitochondria of a healthy human heart). In other embodiments, the databases may include information regarding the metabolome of a variety of organelles (e.g., mitochondria, nuclei, Golgi apparatus, endoplasmic reticulum, ribosomes, cytosol, chloroplasts, etc.) or cells from a particular species from a particular organ in a particular state of health. In another embodiment, the databases may include information regarding either specific organelles or cells from a variety of tissues (e.g., fatty tissue, muscle tissue, nerve tissue, brain tissue, heart tissue, bone tissue, blood, connective tissue, retinal tissue, etc.) from an organism in a health or diseased stated (e.g., the tissue can be from an organism suffering from any disorder known to afflict it). Examples of disorders include neurological disorders, central nervous system disorders, metabolic disorders, cardiovascular disorders, immunological disorders, oncological disorders. In a further embodiment, a database may comprise information regarding compounds of the entire metabolome of a particular species, e.g., human, rat, mouse, dog, cat, etc.

In a preferred embodiment, the database contains information on the small molecule signatures generated from the small molecule profiles obtained from a variety of organisms, organs, tissues, cells or organelles which were treated with or without any agent, toxicant or drug. The database also contains information on the small molecule signatures generated from the small molecule profiles obtained from a variety of organisms, organs, tissues, cells or organdIes obtained from a diseased source or subject, as well as, a healthy reference source or subject. In an additional embodiment, newly generated small molecule profiles from subjects treated with an agent, toxicant or drug of unknown efficacy or toxicity, or profiles from subjects with unknown disease states, can be compared to the small molecule signatures contained within the database to assign the subject as healthy or diseased, or to describe the agent, toxicant or drug as having a particular efficacy or toxicity.

If the database is in electronic form, the program used to organize the database can be any program known in the art which is capable of storing the information in a useful format. The databases of the invention can be organized in such a way that they can be licensed to companies, such as pharmaceutical companies. The databases can then be used for many purposes, such as drug discovery, design, etc.

Agricultural Methods of the Invention

In another embodiment of the invention, the invention includes a method for the identification of agents useful for agriculture, such as for example, insecticides, pesticides, herbicides, and fertilizers.

Plants are an excellent source of small molecules. Many plant small molecules have been shown to have therapeutic benefit. Therefore, in one embodiment, the invention pertains to a library of the small molecules from plant extracts (e.g., extracts from a particular plant or part of plant (e.g., seeds, flowers, berries, roots, sap, leaves, etc.), cells from the plant, organelles (e.g., mitochondria, chloroplasts, nuclei, Golgi apparatus, etc.), cellular compartments, etc. These libraries can also be screened for biologically active molecules using the methods described in previous sections. Furthermore, the plants also can be analyzed using any of the separation or analytical techniques described herein, e.g., mass spectroscopy (MS), HPLC, TLC, electrochemical analysis, refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), Light Scattering analysis (LS) and other methods known in the art.

Furthermore, comparison of plant small molecule signatures could lead to the identification of compounds which are relevant to the plant's resistance of certain diseases or environmental conditions.

In addition, the method also pertains to small molecule signatures and small molecule libraries of plants. For example, the small molecule signatures can be used to determine plant deficiencies of certain compounds, and analyze plant diseases in a method analogous to the comparison of animal small molecule signatures. For example, a small molecule signature can be generated from small molecule profiles of a specific plant ceil, cell compartment or organelle (e.g., chloroplast, mitochondria, endoplasmic reticulum, Golgi apparatus, etc.). Standard plant cell signatures can also be generated. These can be compared to plants in particular disease states to determine which small molecules are present in aberrant amounts in the diseased cells.

In one method of the invention, small molecule signatures generated from small molecule profiles of insect cells, cellular compartments, or specific organelles are compared to small molecule signatures generated from small molecule profiles of insect cells, cellular compartments, or organelles treated with a known insecticide. The small molecule signatures can be compared to identify compounds which are relevant to the insecticide activity. The compounds which are identified as relevant can then be identified to further optimize the insecticidal activity of the compounds.

The term “insecticides” include compounds which kill or other wise limit the reproductive capacity of organisms from the order Isopoda (e.g., Oniscus asellus, Armadillidium vulgare and Porcellio scaber, the order Diplopoda (e.g., Blaniulus guttulatus), the order Chilopoda (e.g., Geophilus carpophagus, Scutigera spec, etc.), the order Symphyla (e.g., Scutigerella immaculata, etc.), the order Thysanura (e.g., Lepisma saccharina, the order Collembola (e.g., Onychiurus armatus), the order Orthoptera (e.g., Blatta orientalis, Periplaneta americana, Leucophaea maderae, Blattella germanica, Acheta domesticus, Gryllotalpa spp., Locusta migratoria migratorioides, Melanoplus differentialis and Schistocerca gregaria, etc.), the order Dermaptera (e.g., Forficula auricularia, etc.), the order Isoptera (e.g., Reticulitermes spp, etc.), the order Anoplura (e.g., Pediculus humanus corporis, Haematopinus spp., Linognathus spp. etc.), the order Mallophaga (e.g., Trichodectes spp. Damalinea spp., etc.), the order Thysanoptera (e.g., Hercinothrips femoralis, Thrips tabaci), the order Heteroptera (Eurygaster spp., Dysdercus intermedius, Piesma quadrata, Cimex lectularius, Rhodnius prolixus and Triatoma spp., etc.), the order Homoptera (e.g., Aleurodes brassicae, Bemisia tabaci, Trialeurodes vaporariorum, Aphis gossypii, Brevicoryne brassicae, Cryptomyzus ribis, Doralis fabae, Doralis pomi, Eriosoma lanigerum, Hyalopterus arundinis, Macrosiphum avenae, Myzus spp., Phorodon humuli, Rhopalosiphum padi, Phylloxera vastatrix, Pemphigus spp., Empoasca spp., Euscelis bilobatus, Nephotettix cincticeps, Lecanium corni, Saissetia oleae, Laodelphax striatellus, Nilaparvata lugens, Aonidiella aurantii, Aspidiotus hederae, Pseudococcus spp., Psylla spp., etc.), the order Lepidoptera, (e.g., Pectinophora gossypiella, Bupalus piniarius, Cheimatobia brumata, Lithocolletis blancardelia, Hyponomeuta padella, Plutella maculipennis, Malacosoma neustria, Euproctis chrysorrhoea, Lymantria spp. Bucculatrix thurberiella, Phyllocnistis citrella, Agrotis spp., Euxoa spp., Feltia spp., Earias insulana, Heliothis spp., Laphygma exigua, Mamestra brassicae, Panolis flammea, Prodenia litura, Spodoptera spp., Trichoplusia ni, Carpocapsa pomonella, Pieris spp., Chilo spp., Pyrausta nubilalis, Ephestia kuehniella, Galleria mellonella, Tineola bisselliella, Tinea pellionella, Hofmannophila pseudospretella, Cacoecia podana, Capua reticulana, Choristoneura fumiferana, Clysia ambiguella, Homona magnanima, Tortrix viridana, etc.), the order Coleoptera (e.g., Anobium punctatum, Rhizopertha dominica, Bruchidius obtectus, Acanthoscelides obtectus, Hylotrupes bajulus, Agelastica alni, Leptinotarsa decemlineata, Phaedon cochleariae, Diabrotica spp., Psylliodes chrysocephala, Epilachna varivestis, Atomaria spp., Oryzaephilus surinamensis, Anthonomus spp., Sitophilus spp., Otiorrhynchus sulcatus, Cosmopolites sordidus, Ceuthorrhynchus assimilis, Hypera postica, Dermestes spp., Trogoderma spp., Anthrenus spp., Attagenus spp., Lyctus spp., Meligethes aeneus, Ptinus spp., Niptus hololeucus, Gibbium psylloides, Tribolium spp., Tenebrio molitor, Agriotes spp., Conoderus spp., Melolontha melolontha, Amphimallon solstitialis Costelytra zealandica, etc.), the order Hymenoptera, (Diprion spp., Hoplocampa spp., Lasius spp., Monomorium pharaonis, Vespa spp., etc.), the order of the Diptera (e.g, Aedes spp., Anopheles spp., Culex spp., Drosophila melanogaster, Musca spp., Fannia spp., Calliphora erythrocephala, Lucilia spp., Chrysomyia spp., Cuterebra spp., Gastrophilus spp., Hyppobosca spp., Stomoxys spp., Oestrus spp., Hypoderma spp., Tabanus spp., Tannia spp., Bibio hortulanus, Oscinella frit, Phorbia spp., Pegomyia hyoscyami, Ceratitis capitata, Dacus oleae, Tipula paludosa, etc.), the order Siphonaptera (e.g., Xenopsylla cheopis and Ceratophyllus spp., etc.), the order Araclinida (e.g., Scorpio maurus, Latrodectus mactans, etc.), the order Acarina (e.g., Acarus siro, Argas spp., Ornithodoros spp., Dermanyssus gallinae, Eriophyes ribis, Phyllocoptruta oleivora, Boophilus spp., Rhipicephalus spp., Amblyomma spp., Hyalomma spp., Ixodes spp., Psoroptes spp., Chorioptes spp., Sarcoptes spp., Tarsonemus spp., Bryobia praetiosa, Panonychus spp., Tetranychus spp, etc.), Pratylenchus spp., Radopholus similis, Ditylenchus dipsaci, Tylenchulus semipenetrans, Heterodera spp., Meloidogyne spp., Aphelenchoides spp., Longidorus spp., Xiphinema spp., and Trichodorus spp.

In another embodiment, small molecule signatures generated from small molecule profiles of insect cells treated with a test compound can be compared to small molecule signatures generated from small molecule profiles of insect cells treated with a known insecticide to determine whether the test compound may be an active insecticide.

The invention also pertains to insecticides comprising one or more insecticides identified by the methods of the invention. In one embodiment, the insecticides of the invention are non-toxic to humans.

The insecticide compositions of the invention, both solids and liquids, may be applied to insect infestations or insect populations by spraying. The methods and equipment needed for a given treatment may be determined by one skilled in the art. Furthermore, methods of the invention described herein may be used to treat insect infestations or populations in dry, moist, or aquatic systems (e.g., the insect-infested area is a flowing or a standing body of water). An aquatic system which is treated with methods of the present invention may be either fresh water or salt water. Furthermore, the insect control compositions of the invention may be applied directly onto a host (e.g., an agricultural crop, a turfgrass).

This invention is further illustrated by the following examples which should not be construed as limiting. The contents of all references and published patents and patent applications cited throughout the application are hereby incorporated by reference.

### EXAMPLES

### Example 1

### Cell Culture Biological Sourcing and Experimental Design

Isolation of Rat Hepatocvtes:

Preheat perfusion and collagenase buffers in a water bath to 37° C. and make sure pH of perfusion buffer is 7.4 and collagenase is 7.6. Oxygenate perfusion buffer for approximately 5 minutes with 95/5% O₂/CO₂. Anesthetize rat with metophane and wipe abdomen with ethanol and shave off hair. Open the abdomen and place a tie previously rinsed in ethanol around the inferior vena cava as well as the portal vein. Cannulate portal vein and begin perfusing with perfusion buffer at 5 mL/min. Immediately cut inferior vena cava, open chest, and cut heart. Tie off vena cava.

Perfuse 50 mL of perfusion buffer through the liver. During this time, prepare the syringe containing the warmed collagenase buffer and perfuse 50 mL of collagenase buffer through the liver until the liver begins to break down. Remove liver from body cavity of rat and place into weigh boat with ice cold wash buffer and loosen cells. Filter cells over nylon mesh.

In a conical tube, bring volume of hepatocyte suspension up to 50 mL with wash buffer and centrifuge at 300 rpm (30 g) for 3 min. Save pellet (hepatocytes) and discard supernatant (Kupffer cells). Wash pellet 3× with 30-40 mL of wash buffer and carefully resuspend hepatocyte pellet after each wash by gently inverting the conical tube. Carefully pipet 15 mL hepatocyte suspension on top of 14 mL 90% Percoll and mix solution to uniformity with a wide-bore pipet tip. Centrifuge 10 min at 500 rpm (50 g) (slow start and finish). Remove media on top containing dead cells, resuspend pelleted cells in 20 mL wash buffer, and wash a final time with wash buffer at 500 rpm (50 g) for 2 min. Resuspend cells in appropriate media and assess cell viability with 0.4% Trypan blue. Add 10 uL Trypan blue to 10 uL of diluted cells and immediately examine under a microscope.

Cell Culture Media and Reagents:

10× Salt Solution: 68 g NaCl, 4 g KCl, 10 g HEPES, 22 g NaHCO₃, 10 g Glucose, and 2 mL 4 N NaOH (8 g NaOH in 50 mL dH₂O). Dissolve in 1 L dH₂O. Adjust pH to 7.4 and filter sterilize.

100× Honnone Mix: Add 296 mg of BSA to 237 mL 0.9% NaCl (1.25 mg/mL) (Solution A). Dissolve 1 mg of Insulin (from bovine pancreas) in 100 μL of 0.1 N HCl (Solution B). Dissolve 7 mg of Glucagon in 100 μL of 0.1 N HCl and add 9.9 mL NaCl/BSA (15 mg BSA in 1 mL 0.9% NaCl) (Solution C). Pre-dissolve 1.35 mg L-3,3′,5 Triiothyronine in 200 μL of 0.1 N NaOH and add 19.8 mL NaCl/BSA (Solution D). Add 53.2 mg Dexamethasone phosphate to 10 mL 0.9% NaCl (Solution E). Add together the following: 25 mL of A to B, then add 84 μL of C and mix. Add the remaining volume of A (212 mL) while mixing. Add 2.4 mL of D and 0.24 mL of E. Filter sterilize. Portion into 20 mL batches and freeze for a max of 1 year. Use 1 mL Hormone Mix per 100 mL medium.

Perfusion Buffer (100 mL): 88 mL ddH₂O, 10 mL10× Salt buffer, 1 mL Heparin (100× stock solution: 62 mg/50 mL 0.9% NaCl), 1 mL Penicillin/Strep (100× stock solution), and 0.2 mL Na-pyruvate (500× stock solution: 1.1 g/20 mL 0.9% NaCl).

Collagenase Buffer (35 mL): 30 mL ddH₂O, 0.35 mL Penicillin/Strep, 3.5 mL Salt buffer, 0.35 mL Hormone mix, 0.7 mL 100 mM CaCl₂ (100× stock solution), and 7 mg Sigma collagenase type IV

Wash Buffer (450 mL):435 mL MEME (with Earle's salts, without L-glutamine, without phenol red), 0.9 mL Na-pyruvate, 4.5 mL Glutamax, 4.5 mL 100× Antimycotic solution, 4.5 mL HEPES (100× stock solution: 2 M), 0.09 mL Aprotinin (stock solution: 2000 U/L), 0.45 mL Dexamethasone phosphate (stock solution 1000×: 3 mM), and 1.24 mL Eli Lilly Humulin R

10× HBSS: 80 g NaCl, 4 g KCl, 2 g MgSO₄.7H₂O, 0.6 g KH₂PO₄, 10 g Glucose, and 0.91 g Na₂HPO₄.7H₂O. Dissolve in 1 L dH₂O, adjust pH to 7.4, and filter sterilize

Plating and Culture Media: 1^{st} 24 hours (500 mL): 434 mL WE, 5 mL Glutamax, 5 mL Antimycotic solution, 5 mL HEPES, 0.5 mL Dexamethasone phosphate, 1.38 mL Eli Lilly Humulin R, and 50 mL 10% Fetal Calf Serum.

Culture Media: post 24 hours (500 mL): 484 mL WE, 5 mL Glutamax, 5 mL Antimycotic solution, 5 mL BEPES, 0.5 mL Dexamethasone phosphate, and 1.38 mL Humulin R

90% Percoll (14 mL): 1.4 mL 10× HBSS, 12.6 mL 100% filter sterilized Percoll, pH 7.0.

Signature Generation in Cultured Cells:

To generate signatures for the efficacy and toxicity of compounds, the drugs are applied to the desired cell type, at several concentrations and over a time course. A concentration is chosen that is estimated to be near the concentration achieved in vivo following dosing of an animal or human with the compound, and bracketed by additional doses several-fold above and below that level. For each of these samples, between 5×10⁴ and 5×10⁵ cells are generally used. Cells are generally plated at a standard density prior to the experiment, such that they will be in early plateau phase at the beginning of the dosing experiment. For some drug classes, however, it may be more appropriate to dose cells in exponential phase growth or post-confluence, and the density of cell seeding and or duration of the pre-dosing growth phase are adjusted accordingly.

The dosing experiment can be conducted in normal medium, or in serum-free medium, or in a balanced buffer solution, as appropriate for the cell type and compound. Usually, cells are plated in multiwell plates; each well provides a medium sample and a cell extract sample for a particular combination of dose level and exposure time.

Samples produced in the dosing experiment include extracts of cells, as well as of the growth medium that contained them. The latter is useful in generating signatures because cells absorb compounds from the medium, and secrete many metabolic products into the medium. Cells with physiology that has been altered by exposure to drugs or toxins may absorb or secrete biochemicals at different rates, or export biochemicals not normally observed in the medium.

For example, plate CHO-K1 cells in the wells of three 24-well-plates at a density of 2-3×10⁴ cells/cm² in DMEM and grow cells for 2 days. Add the test compound to 12 wells at each of 7 dose-levels (e.g., 0, 0.1 ng/ml, 1 ng/ml, 10 ng/ml, 100 ng/ml, and 1 μg/ml). The zero-dose control cells are mock-treated, by adding to the medium the solvent for the drugs in the experiment (typically water, but sometimes DMSO or an alcohol.). At intervals (e.g., 1, 4, 8, and 24 hours), medium is removed from 3 wells containing each dose-level and stored for sampling. A small sample of the removed medium is placed at −80° C. for later extraction and analysis by mass spectrometry. The well is washed twice with HBSS to remove residual medium from the cells adhering to the well. The cells are lysed into extraction buffer directly in the wells, and the lysate is stored at −80° C. for completion of the extraction process. To generate signatures for one compound, an experiment might include 3 replicate cultures of each of 4 to 6 dose levels, over 4 to 6 time intervals of drug treatment, resulting in 60 to 126 samples.

Drug Screening for Efficacy Signatures:

To screen compounds of unknown efficacy or toxicity for matches to signatures present in the database, essentially the same experiment is performed as for signature generation.

Because the dose levels that may result in efficacy or toxicity are not known, it may not be possible to use the concentration to which cells are exposed during standard therapy in vivo as a starting point. Therefore all drugs will be dosed at several concentrations, but the range covered will be wider than in the signature generation experiment.

Panels of cells are often used in screening drugs, since different cell types are expected to respond differentially to dosed compounds. Thus a screening experiment typically includes all the cell types for which meaningful signatures have been developed.

### Example 2

### Animal Sample Biological Sourcing and Experimental Design

Rat Strains and Disease Experimental Systems:

Random bred, male and female rats are purchased from Charles River Laboratory (Boston, Mass.). Animals are segregated by sex, in plastic cages with stainless steel tops, two per cage, and acclimated for 4 to 7 days in temperature (70-78° F.) and humidity (30-70% RH) controlled rooms with a 12-h light cycle. Food (Purina Certified Rodent Chow®-5002) and water are provided ad libitum. Sprague-Dawley rats are used for generating efficacy and toxicity signatures in normal healthy animals. Alternative rat strains, such as Hans-Wistar, may also be used.

Drug and Toxicant Dosing:

Dosing is accomplished using a variety of methods depending on the chemical characteristics and pharmacodynamics of the drug or toxicant. Drugs are generally dosed orally (for example by gavage) or by injection (for example intraperitoneally). A variety of substances are used as vehicle for the drug or toxicant. Examples include corn oil, dimethyl sulfoxide, phosphate-buffered saline, 0.9% saline, sterile water, and powdered chow.

Sample Choice and Collection:

Disease, therapeutic agents, and toxicants, alter the biochermical constituents of the target organ(s) they affect, resulting in characteristic signatures that are detected by mass spectrometry. It follows that the choice of biological sample to signature is dictated by the specific target organ(s) affected. However, blood and urine in general also yield informative signatures that reflect the biochemical alterations of target organ(s). Collection of blood and urine is relatively non-invasive and can be performed multiple times on an individual animal, offering advantages over other types of tissue samples (e.g. liver), which require destruction of the animal. The protocols described herein utilize blood and urine for the generation and screening of efficacy- and toxicity-signatures. However any biological fluid or tissue may be used, in addition to blood and urine, as appropriate for the specific requirements of the experiment. In addition to blood and urine collection, soft tissues can be used.

Blood: Blood (10 to 300 μl) is collected by saphenous vein puncture, as outlined by Hem et al. (Laboratory Animals 32: 364-368, 1998), and extracted immediately as described below. Alternatively, blood samples are spotted onto S&S Grade 903 filter paper (Schleicher and Schuell), dried, and stored for later extraction (Chace et al. Clin Chem. 47:1166-82, 2001). The dried blood spot is subsequently recovered from the filter paper using a hole-puncher, placed in a microtiter well, and extracted as described below. Alternatively, the blood is collected directly into a heparinised tube, and the plasma separated by centrifugation, prior to extraction. Alternatively, the blood is allowed to clot and the serum separated by centrifugation, prior to extraction. Blood, plasma, or serum samples may be stored at −80° C. prior to extraction.

Urine: Urine is collected using a catheter inserted into the urinary bladder and aliquoted into a microtiter plate prior to extraction, as described below. Alternatively, urine samples are spotted onto S&S Grade 903 filter paper (Schleicher and Schuell), dried, and stored for later extraction. Other methods of urine collection, i.e. using metabolic cages or manual expression, can also be used.

Soft tissues: Rats are euthanized by carbon dioxide inhalation. Harvested tissues are rinsed with PBS (20 mM sodium phosphate, 150 mM sodium chloride, pH 7.4) and weighed. Tissues are either processed immediately as described below or are flash frozen with liquid nitrogen and stored at −80° C. for extraction at a later time.

Experimental Designs:

Efficacy-Signature Generation: Compounds of known efficacy are used for dosing. Signatures are generated using either normal healthy rats or diseased rats appropriate for the therapeutic class under study. For each drug, three to seven dosage levels, including a zero dose (i.e. vehicle alone) are used. The appropriate dose range, number of doses/animal, and duration of dosing, are determined for each drug depending on its pharmacodynamic and pharmacokinetic properties. Each treatment (i.e. dosage level) is administered to five to ten rats. Blood and urine samples are collected, as described above, from each animal prior to initiation of treatment and subsequently over a time course that coincides with the therapeutic affects of the administered drug. Blood and urine samples are processed for mass spectrometry as described below. At termination, tissue samples from organs that are known targets of the administered drug are harvested for biochemical profiling. Appropriate clinical observations, clinical chemistry panels, and histopathology are used to assess the therapeutic actions of the treatments and to correlate these with biochemical signatures. Table 1 lists drugs that are used to generate efficacy signatures for two major therapeutic classes, NIDDM and RA/I.

Efficacy-Signature Screening: Compounds of unknown efficacy are used for dosing. The source of compounds may include combinatorial chemical libraries used in early drug discovery phases, custom synthesized chemicals used in lead optimization phases, or compounds already approved by the FDA for other medical indications. Experimental design is similar to that of signature generation. In order to increase throughput, fewer doses and sample collection time points may be used. Either normal healthy rats or the appropriate diseased experimental system may be used. In a typical design, five animals are treated using a single dose level, determined as the maximum tolerated level for that compound. If multiple compounds are screened simultaneously using the same dosing vehicle, a single zero-dose vehicle-control group can be used. Blood and urine are collected at three time points, 0, 1×, and 2× the time of prototypical signature onset, and appropriate tissues are harvested at termination, for biochemical profiling. Compounds that produce a biochemical signature that matches or partially matches the efficacy-signature are subjected to confirmation testing, using more dosage levels, sample collection time points, and varying the duration of treatment. Compounds that have a confirmed efficacy-signature are further evaluated for clinical efficacy in the appropriate disease experimental system (see above), using clinical observations, clinical chemistry panels, and histopathology.

Toxicity-Signature Generation: Compounds of known toxicity are used for dosing. Compounds are selected depending on the class of toxicity being investigated. Toxicity classes may be defined by mechanism-of-action or by target organ. Table 2 lists various known toxicants and the target organs affected by the compound, which may be used for the purpose of toxicity-signature generation. Three to five dose levels (including a zero-dose vehicle control) are used per compound. Five to seven normal healthy rats are treated per dose level. Doses are administered daily for up to 14 days. Blood and urine samples are collected from each rat at 0, 1, 2, 4, 8, and 14 days from the initial dosing date for the purpose of biochemical profiling. At termination, tissue samples from organs that are known targets of the administered toxicant are harvested for biochemical profiling. Toxic actions of the treatments are monitored over the course of the experiment using clinical observations and standard clinical chemistry panels, whereas histopathology is determined upon termination. Clinical observations include weight, behavior, and mortality. Hematological parameters include white blood cell count, hemocrit, erythrocyte count, reticulocyte count, mean cell volume and concentration, and total and differential white blood cell counts. Clinical chemistry parameters on serum or plasma include albumin, bile acids, bilirubin, cholesterol, glucose, urea nitrogen, creatinine, creatinine kinase, a-glutathione-S-transferase, glutamate dehydrogenase, sorbitol dehydrogenase, total protein, triglycerides, aspartate aminotransferase, alkaline phosphatase, alanine aminotransferase, and globulins. Urine parameters include bile, creatinine, and nitrite. Histopathological examination may be performed on liver, kidney or any other relevant tissues; tissues are fixed, sectioned, stained with hematoxylin and eosin, and observed by light-microscopy. The observed responses to toxicity are later correlated with biochemical signatures, such that the latter may be used as a predictor of toxic response.

Toxicity Signature Screening: Compounds of unknown toxicity are used for dosing. The source of compounds and dosing design are largely as described above for efficacy screening. In fact, the very same biochemical signatures produced during efficacy screening may be used to search for toxicity-signatures. That is, compounds are screened for both efficacy- and toxicity-signatures in a single experiment. Alternatively the experiment can be designed for the purpose of toxicity-screening without regard to efficacy. Compounds that produce a biochemical signature that matches or partially matches the toxicity-signature are subjected confirmation testing, by using more dosage levels, sample collection time points, and by varying the duration of treatment. Compounds that have a confirmed toxicity-signature are further tested for clinical signs of toxicity by using clinical observations, clinical chemistry panels, and histopathology.

### Example 3

### Human Biological Sourcing and Experimental Design

Dosing experiments

Subjects not taking other medication are dosed with drugs at several dosages up to the maximum allowable dose, and samples of blood, blood fractions, or urine are taken over a time course. For some therapeutic areas or drugs, samples of other fluids, such as synovial fluid or cerebro-spinal fluid, may be used. Typically, at least 10 subjects are treated at each dose-level for each compound studied. Blood pressure, standard clinical chemistries, and any drug-specific parameters are measured at each sampling point, to establish correlation between biochemical profiling results and traditional indicators.

Two dosing/sampling schemes are used:

Acute dosing: In this scheme, the subject is given a single dose, and samples are taken over a short time course following dosing, including a time that is expected to include return to baseline. The exact time course followed will depend on the known pharmacokinetics of the compound. For example, 4 patient groups are established, each with 20 patients. Each group is dosed at 0, 0.1×, 0.3×, or 1× the maximum allowable dose. Samples of whole blood and urine are obtained from each patient pre-dosing, and at 30′, 1h, 2h, 4h, 8h, and 24h post-dose.

Chronic dosing: This scheme is appropriate for generating signatures for maintenance drugs, which often must be taken for a longer time to achieve disease modification. In this scheme, compounds are dosed repeatedly, but sampled at a small number of time points. For example, 20 patients and 20 normal healthy volunteers are randomized to placebo and drug groups. Each patient is dosed daily with placebo or drug. Samples of urine and whole blood are obtained pre-dose and after 14 and 30 days of treatment.

Sample collection:

Blood: Whole blood, serum or plasma are analyzed.

Whole Blood: Following a finger-stick with a sterile lancet, up to 200 ul of capillary blood are spotted onto a 3 mm disc of S&S Grade 903 filter paper (Schleicher and Schuell), and dried. The disc is then transferred to a well of a deep-well microtiter plate for extraction using methods below.

Serum: Whole blood (50 ul to 5 ml) obtained via finger stick or venous blood draw is allowed to clot at room temperature. Serum is removed from above the clot. Chelating agent and antioxidant are added (EDTA, final concentration 0.4 mM; TEMPO, final concentration 0.8 mM). The sample is extracted immediately, or stored at −80° C. until extraction.

Plasma: Whole blood is collected into an anti-coagulant (heparin or citrate/EDTA). Cells are removed by centrifugation (xg for y min). The plasma layer above the cells is removed to a new tube containing chelating agent and antioxidant are added (EDTA, final concentration 0.4 mM; TEMPO, final concentration 0.8 mM). The sample is extracted immediately, or stored at −80° C. until extraction.

Urine: Urine is collected using a “clean catch” method. The urine is stored as soon as possible at −80° C. Immediately upon thawing, a chelating agent and an anti-oxidant are added to the urine (EDTA, final concentration 0.4 mM; TEMPO, final concentration 0.8 mM). Urine is extracted as described below.

### Example 4

### Mass Spectrometry

MS Platform Descriptions:

Two different mass spectrometry (MS) platforms are contemplated including Targeted using multidimensional HPLC/MS/MS and Discovery using flow injection analysis (FIA) and time-of-flight (TOF) MS. For each platform, a biological sample must first be prepared by an extraction method.

Sample Preparation:

The biological fluids or cells (e.g. a suspension of in vitro cultured cells, blood, urine etc.) arrayed in a 96-well plate are mixed with an equal volume of extraction solvent (e.g. 90/10 Acetonitrile/water, 1% trifluoroacetic acid) and vortexed for 60 seconds. If using soft-tissues (e.g. liver), the tissue is homogenized at 4° C. using a Teflon-on-glass or other appropriate homogenizer in an equal volume of extraction solvent. The resulting solution or homogenate from the above steps is centrifuged at 3,000 g for 15 minutes to remove precipitated proteins and other macromolecules. 100 μl of the supernatant is transferred to a new 96-well plate and dried under Nitrogen The dried sample is then stored at −80° C., until ready for analysis, at which time it is reconstituted with the Internal Standard solution (Stable isotopic and/or deuteriated compounds e.g., Glucose-d7, Valine-d8, glycerol-d8 in 50/50 acetonitrile/water). Alternatively, a biological fluid can be used directly, after dilution with the Internal Standard solution.

The standard extraction protocol described above can be used for either MS platform. In addition, the discovery platform may use additional extraction protocols to extract a wide range of biochemical compounds. One example would be to use chloroform/methanol as a solvent in addition to acetonitrile/water.

Targeted Platform

The targeted platform detects the presence of molecules from a defined list of biochemical compounds and only from this list. Other molecules present in the sample are not detected. This platform is used to create signatures whose components are biochemical compounds that can, in combination, distinguish between classes of samples. Because the identities of the compounds are known, the composition of signatures can be subject to biological interpretation.

There are seven components to the platform: 1—8 HPLC pumps used to deliver liquid phases; 2—A 4-injector autosampler for controlling sample injection; 3—up to four different HPLC columns for separation; 4—A switching valve used to control column to MS transfer; 5—An LC/MS interface such as electrospray (ES), atmosphere pressure chemical ionization (APCI) for connection of BPLC and MS; 6—A triple quadrupole mass spectrometer for compound separation and identification; 7—A computer for instrument control and data acquisition.

The columns are selected from the following types: Luna phenyl-hexyl reverse phase (RP), Synergi-Hydro RP, Luna amino normal phase (NP), and a Luna C18 RP. The combination of column type and mobile phase is selected in order to optimize the separation of the biochemical compounds to be detected by the mass spectrometer. In particular, it is desirable to use the columns to separate compounds (desired or interfering) that have very similar masses of both parent and daughter ions—different classes of compounds are targeted to each of the columns.

The column switching valve allows staggered injection into the multiple columns, and the effluent from the different columns to be analyzed sequentially in a single run. This way data from 4 columns can be captured from single sample on a single mass spectrometer, rather than needing 4 separate runs. Compounds with distinct masses but similar retention times can be separated by the mass spectrometer. The targeted compounds (Table 3) are each detected by the MS throughout the run to produce a series of mass chromatograms.

Mass Chromatogram Processing

Biochemical Compound Identification: In order to quantify a single desired biochemical compound, the triple quadrupole mass spectrometer (FIG. 1) combines two mass filters and a fragmentation step. The first quadrupole acts as a mass filter and only allows ions of a particular mass/charge ratio to proceed further into the second quadrupole. This second chamber acts as the collision cell where the filtered molecules are fragmented with gas molecules and with a source of electrons. This fragmentation causes each parent biochemical molecule to fragment in a predictable manner producing daughter ions of a particular mass. The third quadrupole acts as a second mass filter and only allows the desired daughter ions to pass through to the detector. Thus the combination of the two mass filters allow for quantitation of only molecules with the desired mass/charge ratio that produce daughter ions of the desired mass. In most cases this will detect only a single compound. Distinct biochemical compounds that have identical parent and daughter masses will be ambiguous, and for those situations, it may be possible to use the initial step of liquid chromatography to separate the molecules by retention time.

In order to detect and quantify 200 target compounds in a single mass spectrometer run, the parent and daughter ion masses are programmed into the machine. The two mass filters rapidly cycle through these mass combinations, detecting each of the target compounds as the sample comes off the columns.

Biochemical Compound Quantitation: After peak identification, the amount of each compound must be calculated. This is achieved by the step of peak integration. The area under the peak for each of the target compounds is calculated using the AB Analyst software. These values are then scaled by the area of the internal standard peak, producing a relative peak area ratio.

QC: In addition to standard processing, each sample is run through a suite of QC procedures which examine (among other things) retention times, and peak areas for internal standards for indications of problems with the LC/MS process. In addition, individual peaks can be flagged for manual examination if parameters (such as for peak shape) exceed normal bounds.

Platform Validation and Tuning

The above sections describe the process for running the mass spectrometry platform in production mode. However, in order to reach this point, a great deal of optimization and tuning needs to be done. The general process for developing the platform that can detect hundreds of biochemical compounds is described herein.

The first task is to identify the parent and daughter ion masses that can be used to uniquely identify a particular molecule from the collection of several hundred compounds being targeted. Since many molecules of biological interest have very similar masses and structures, the daughter ions produced upon fragmentation also have similar masses. Using pure standard solutions of each targeted compound, a signature of possible daughter ion masses is generated by varying the energy levels used for fragmentation in the second quadrupole. These signatures are then used to identify daughter ions that allow compounds with similar parent masses to be distinguished. Once the desired daughter ions are identified, the energy level that maximizes the amount of that daughter ion is selected.

The daughter ion signatures collected above can also be used to populate a compound identification database to assist in identification of interesting peaks from the discovery platform, described in detail below.

As detection of each compound is being optimized, the separation of the compounds must also be optimized. If two compounds can't be cleanly distinguished by modifying fragmentation energy and daughter ion masses, then chromatographic separation is relied upon. Mobile phase gradient testing is used to optimize the separation of key compounds, in addition to the use of the three different columns. For example, amino acids are initially assigned to a phenyl-hexyl RP column; prostaglandins and leucotrienes are assigned to a C18 RP column; and sugars, sugar phosphates and organic acids are assigned to an amino NP column. Then, all other biochemical compounds are tested on each of these three columns for compatibility with each of the sets of compounds already assigned. Any that fail to be added to the initial three columns can be added to the fourth column (such as a Synergi-hydro RP column). A fourth column can also be added to target small proteins and peptides.

The other important component of this platform is reducing the time of the overall run. A short run is essential for a high throughput operation. However, since detecting each compound takes a finite amount of time, there is an inverse relationship between the number of compounds that are being measured simultaneously and the number of data points that can be collected. The key to this optimization is that not all 200 compounds need to be measured for the entire length of the run. Different sets of compounds are targeted to each of the columns, and so detection for each set can be limited to the appropriate fraction (period) of the overall run. This can be further optimized by the knowledge that each compound has a specific retention time coming off the column, and detection can be limited to a window around that time. This compound-specific retention time varies with the each type of biological sample (e.g. blood, urine), adding a further variable that must be taken into account when setting up the platform.

Discovery Platform

This platform has the ability to measure thousands of molecules in a mixture due to a high mass resolution, but does so on the basis of mass/charge ratio alone. In contrast to the Targeted platform, there is no chromatography separation step, or ion fragmentation step to assist in distinguishing same-mass molecules. As a result, the data will not allow for the direct identification of individual biochemical compounds.

There are five components to the platform: 1—HPLC pumps used to deliver liquid phases; 2—An autosampler for controlling flow injection analysis; 3—An LC/MS interface such as electrospray (ES), atmosphere pressure chemical ionization (APCI) for connection of HPLC and MS; 4—A time-of-flight (TOF) mass spectrometer; 5—A computer for instrument control and data acquisition.

The TOF MS instrument measures the quantity of ions across a defined range of mass/charge rations to produce a mass spectrum.

Mass Spectra Processing

Spectra Alignment: Due to the high mass resolution of the TOF mass detector, slight variations can occur in the mass/charge ratios detected by the instrument for a given molecule. To enable comparison across multiple samples, the spectra must first be aligned. This alignment step, which involves both translation and compression/expansion of the spectra, results in an assignment of an ID to each peak in the spectrum. This identifier alone does not allow identification of the biochemical compound(s) present at that mass/charge.

Identification of Biochemical Compounds: The value of the mass/charge ratio is often the only information available for a given spectrum peak. If needed, further experiments can be designed to elucidate the likely identification of the biochemical compound that makes up that peak. For example, for a given peak of interest, the same sample could be run through the triple quadrupole instrument. The mass observed in the QTOF would be used as the first mass filter, and a range of energies would be used for fragmentation. The pattern of daughter ions generated at each energy can be compared to all the data collected during optimization of the targeted platform to aid in compound identification.

QC: The principles of quality control for the discovery platform are similar to those for the Targeted Platform. The intent is to identify samples that produce data that is outside of the normal ranges expected from the platform in order to provide feedback to the MS operation.

### Example 5

### Bioinformatics

The data output by Mass Spectrometry is subjected to a number of analytical methods with the goal of either generating a signature (such as efficacy, toxicity, or disease) from a group of samples, or matching unknown samples to a previously generated signature. The following steps describe the general process of data analysis.

Data Capture from Mass Spectrometry

For the Targeted platform, the initial data captured by the MS instrument is in the form of intensity vs. retention time peaks for each targeted molecule. To obtain the relative abundance of the targeted molecule, the area under the target peak is calculated (peak integration) and divided by the area under the peak for the internal standard molecule. The resulting output is a table of relative abundance values, one per biochemical compound targeted by the platform.

The Discovery platform has no peak integration step since there is no chromatographic separation prior to the TOF analysis. Each mass detected has an intensity and is treated independently for analysis. The output from the discovery platform is a table of relative abundance values, one for each mass detected by the instrument. No biochemical identification is associated with these masses. The resulting table will be much larger than that for the targeted platform because all compounds will be detected, not just the targeted ones.

Generating Summary Dose/time-response Signatures

For each drug or toxicant experiment, there are samples collected from multiple times and doses (and replicates of each). The output derived from the MS analysis of a single sample is defined as a biochemical signature. Thus each experiment generates multiple biochemical signatures which must be collected together and analyzed as a group. Signatures with treatment are compared to a contrasting control signature (e.g. with no treatment at the same time, or at time zero and the same dose) to derive the changes in abundance for each biochemical compound. Dose-time response surfaces can then be constructed to represent the biochemical changes that occur during the experiment.

For further analysis, the time/dose data will be used in one of two ways. The first approach would be to use the time and dose information to identify an optimal dose as well as a steady-state time point for which to use a signature as a representative for the treatment in question for clustering and classification, see below. The second approach begins with a per-biochemical parameterization based on the entire time series (e.g. measurement rise-time, level of maximum response) followed by clustering of samples where the features are the parameters for each biochemical (rather than the measurements themselves).

Classification/Clustering

In order to discover signatures for a class of drugs, toxicants or diseases, signatures from the appropriate experiments are analyzed with a classification algorithm such as hierarchical clustering, relevance networks or classification trees. These methods may be preceded by appropriate dimension-reduction techniques such as projection pursuit in order to minimize the effect of coordinately acting biochemical compounds. The goal is to obtain a grouping of samples (such as those derived from a given class of drugs, or those from subjects with similar diseases) based on signature similarity so that both the within-group homogeneity and between-group separation are high. A training set of samples with known associated toxicity, efficacy and disease-state attributes will be used for classification of new uncharacterized samples.

The effectiveness of any given classification approach is measured by the ability of the algorithm to group an unknown signature into the correct class. Once a suitable clustering has been obtained, the contribution of each biochemical compound to that cluster discrimination can be measured to identify those biochemical compounds that are most useful as a biochemical signature for the class (a signature being the informative subset of biochemical compounds from a set of signatures).

The identification of a limited number of biochemical compounds that together provide the bulk of the discrimination power (i.e. the components of a signature) can then be used to develop a more compact/economical diagnostic test.

Signature Database and Screening

Each experiment's results (including time-dose response signatures, clustering parameters and signature components) are stored in a central results database that enables a single signature search. Thus, the signature obtained from a single treatment-control sample-pair can be used to find other treatments/diseases that cause a similar pattern of biochemical changes. This is particularly important in screening a large number of patients for disease signatures, or a large number of drugs for efficacy or toxicity.

### Example 6

### Information Management

All phases of the experimental process from receipt of animals, cell lines and experimental design, through to data analysis and signature creation are enabled by a comprehensive information management database system. Objects tracked by this system include experimental samples such as biosamples (blood, urine, cells) and MS samples (biosamples after extraction); containers such as cages, tubes, 96-well plates, racks, freezers; and events such as receipt, dosing, sampling, extraction, drying, freezing, storage etc. Objects are labeled with barcodes which allow tracking through the laboratory with the use of handheld barcode scanners. The data model for the database allows for a hierarchical organization of samples into experiments and projects and thus facilitates easy data extraction for the purposes of analysis.

The Information Management Database System is a Web-based Java application that follows the J2EE architecture and its latest specifications. The Web tier uses the Struts framework, which is a model-view-controller framework for constructing web applications with Servlets and Java Server Pages (JSPs). The middle tier uses Enterprise Java Beans (EJBs), both Sessions Beans and Entity Beans, to convert clients requests into Database calls and apply any necessary business logic. The application server provides a container where the EJBs reside at this time, the application Server is Weblogic 7.0). Last, the persistence (data) tier is contained in an Oracle 9i Database. This architecture separates clearly the application in three layers, which results in improved performance and code maintenance compared to the traditional two-tier architecture. In addition, the Struts and J2EE frameworks allow fast response to functionality changes and higher productivity, since a large part of the application framework is already taken care of by the Struts classes and the EJB container.

### Example 7

### Signature Generation

In this example experiment, ten rats were dosed orally for each treatment. Ten rats were treated with Hydrazine, ten were treated with ANIT and ten were treated with vehicle alone (control). Urine and blood samples were taken at 0, 6 and 24 hours post dose and samples were analyzed by mass spectroscopy. There were 174 analytes which were organized into three assays (amino acids, sugars/nucleotides, and organic acids). Data disclosed herein represents analysis of the amino acid assay (44 analytes) on serum from the hydrazine and control treatments.

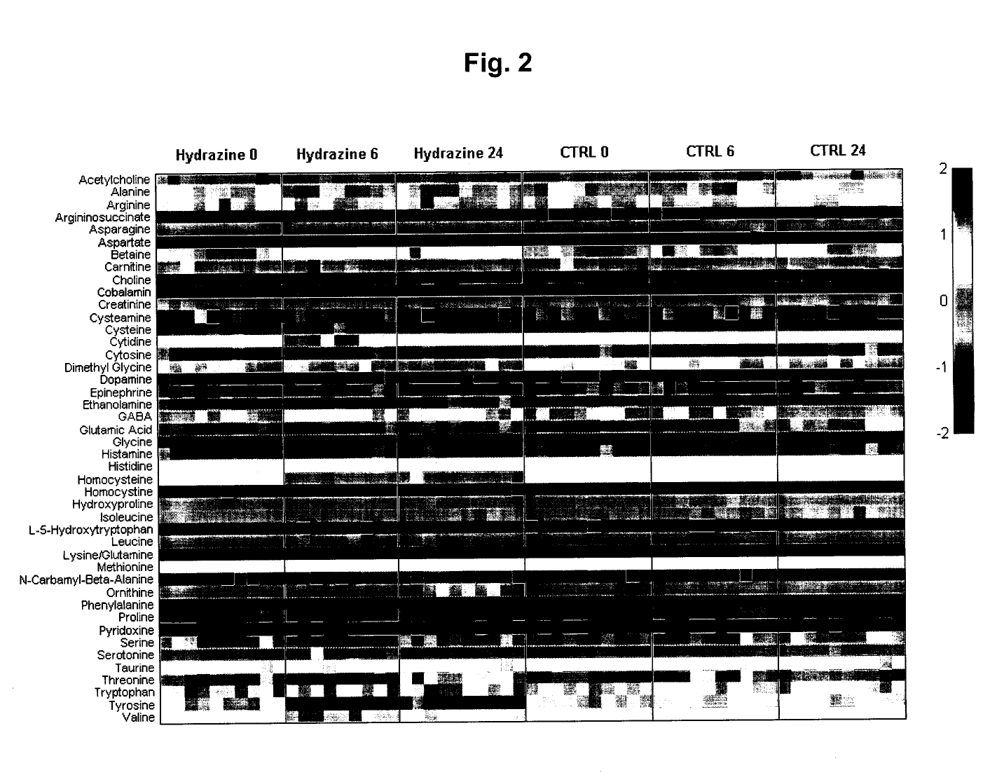

FIG. 2 shows a matrix of N analytes×M samples. For easy visualization, the columns (samples) are sorted by treatment (hydrazine or control) and then time point (0, 6, and 24). The boundaries (black vertical lines) group rats that are considered replicates (that is, subject to the identical treatment). Each rat for a particular treatment participates in all time points. Each cell in this colormap is the measurement of the ith analyte of the jth sample. The numbers are initially peak area ratios (area under the analyte peak divided by the area of the sample's internal standard). Each column in the above matrix is scaled by its median value to account for global offsets in intensity per sample.

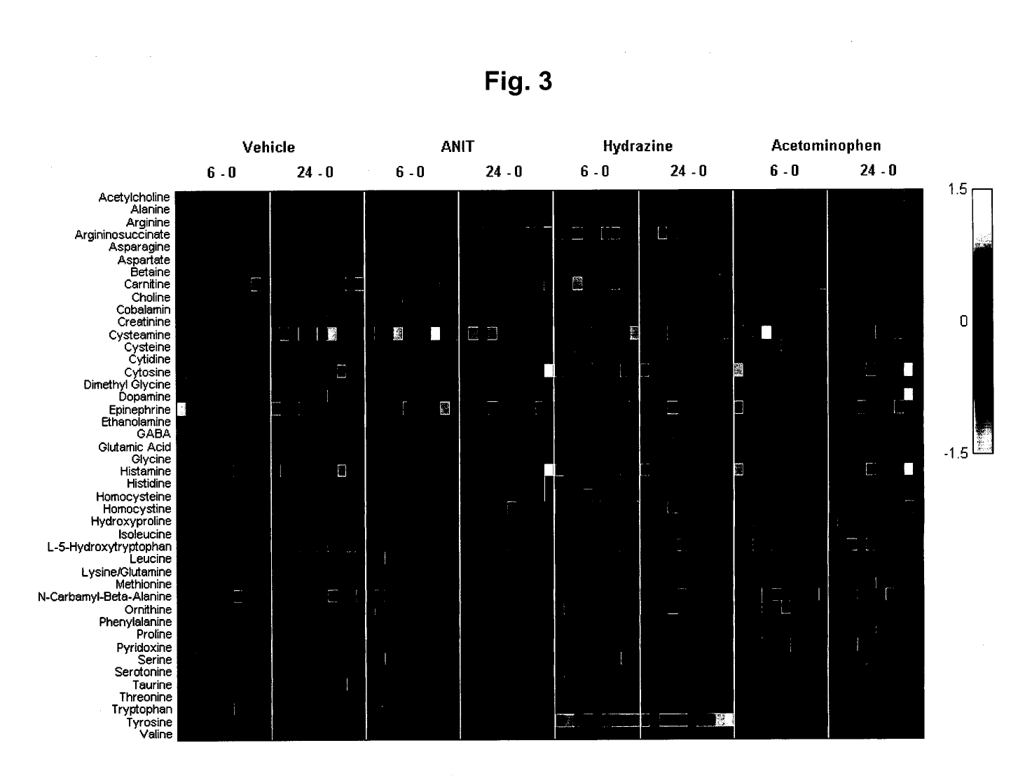

FIG. 3 shows that within each treatment, and for each rat, the measurement at the first time point is subtracted from the measurements of the later time points (in this case, 6 and 24). This converts the measurements from relative abundance measures to changes relative to time zero. The colormap used here is different (black in the middle for “0”) to reflect the fact that measurement changes are being depicted. This plot shows results for ANIT and Acetominophen in addition to hydrazine which was shown in FIG. 2.

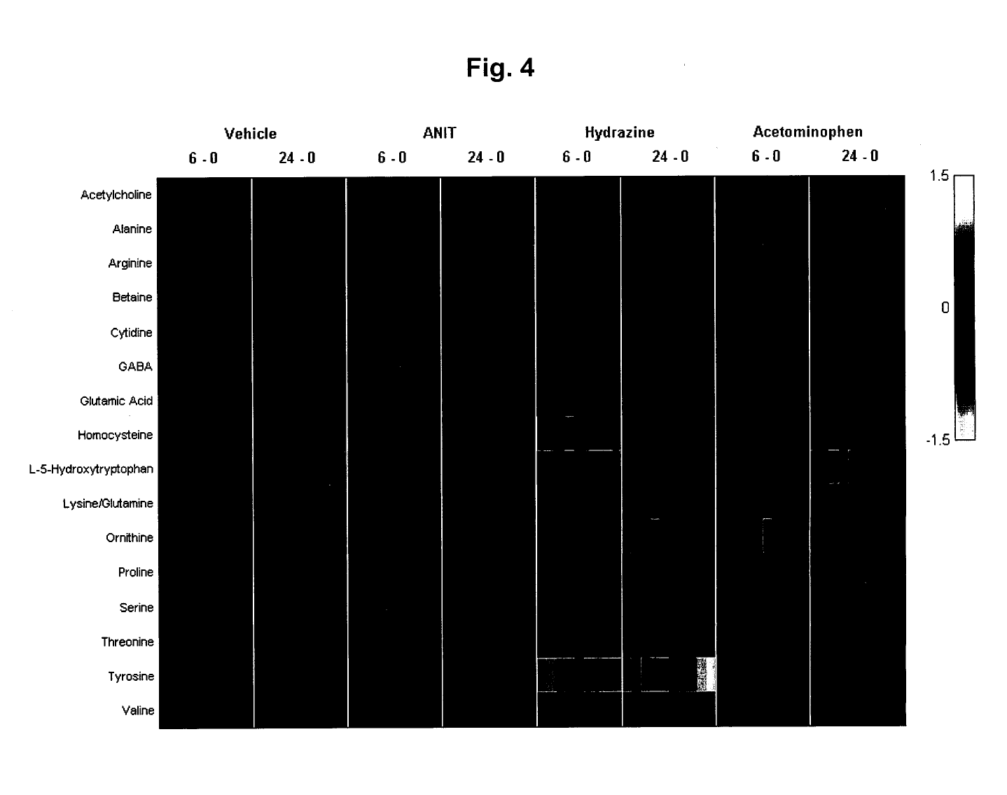

FIG. 4 indicates that to narrow down the set of analytes that are informative for a signature, a score that compares the level of within treatment-time variability with across treatment variability (in this case, the F statistic) is used to preserve rows (analytes) for which the variability of the measurements (change relative to time zero) within a treatment-time is low realtive to the variability across all measurements.

Next, all replicates within a treatment-time (that is, all of the cells in a given row bounded by a pair of adjacent white lines) are averaged. The measurements for the corresponding times for the vehicle (control) are subtracted from those for the hydrazine (or other treatment). This subtracts out the effect of vehicle from the treatment response.

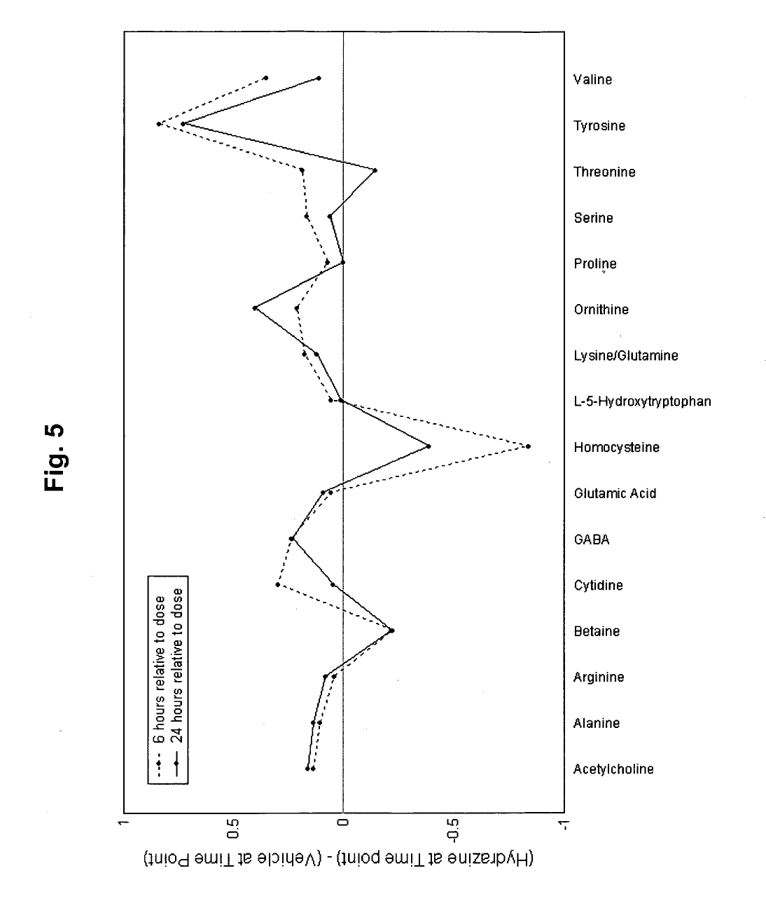

The hydrazine signature in FIG. 5 reflects the changes in the component analytes at 6 hours (dashed) and 24 hours (solid) for hydrazine relative to control (mock treatment). The 16 analytes displayed combine to make the hydrazine and the ANIT signatures. Some of the 16 analytes displayed are not informative for hydrazine.

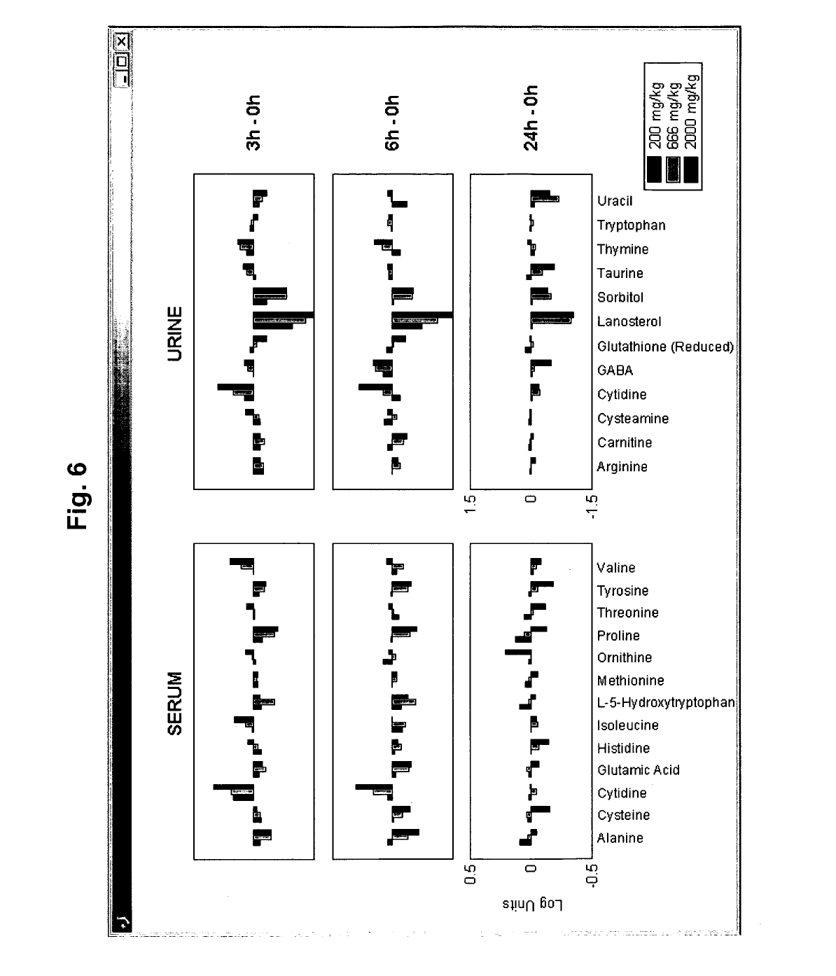

FIG. 6 shows that the signature component analytes in serum and urine (with the exception of Cytidine) are completely different. Many decay back to zero at 24h and a dose-response is evident in many analytes (e.g. see cytidine, lanosterol, proline).

TABLE 1
Drugs to be used for generating efficacy signatures
for non-insulin-dependent diabetes (NIDDM) and
rheumatoid arthritis/inflammation (RA/I).

| Therapeutic Class | Drug |
|---|---|
| NIDDM | Chlorpropamide |
| NIDDM | Tolbuamide |
| NIDDM | Tolazamide |
| NIDDM | Acetohexamide |
| NIDDM | Glyburide |
| NIDDM | Glipizide |
| NIDDM | Glimepiride |
| NIDDM | Pioglitazone |
| NIDDM | Rosiglitazone |
| NIDDM | Metformin |
| NIDDM | Acarbose (Precose) |
| NIDDM | Miglitol (Glycet) |
| NIDDM | Repaglinide (Prandin) |
| RA/I | Aspirin |
| RA/I | Acetaminophen |
| RA/I | Ibuprofen |
| RA/I | Indomethacin |
| RA/I | Peroxicam |
| RA/I | Tometin |
| RA/I | Rofecoxib |
| RA/I | Celecoxib |
| RA/I | Valdecoxib |
| RA/I | Methotrexate |
| RA/I | Dexamethasone |

TABLE 2
Known toxins to be used for toxicity signature generation
studies, and the organs known to be targeted.

| Toxicant | Target organ/organ system |
|---|---|
| 2,2′,4,4′,5,5′-hexachlorobiphenyl (PCB-153) | liver |
| 2,3,7,8-tetrachlorodibenzo-p-dioxin (TCDD) | liver |
| 2-bromoethylamine (BEA) | kidney |
| 3-methylcholanthrene | liver |
| 4-aminophenol (PAP) | kidney |
| acetaminophen | kidney |
| adriamycin | kidney cortex, liver, heart |
| allyl alcohol | liver |
| amiodarone | cardiovascular system; liver; ocular & visual system; |
| | reproductive system; respiratory system |
| amphotericin B | blood; cardiovascular system; kidney; liver |
| Aroclor 1254 | liver |
| Aroclor 1260 | liver |
| arsenic | liver |
| aspirin | cardiovascular system; kidney |
| astemizole | cardiovascular system |
| benzene | blood |
| cadmium | kidney; liver; nervous system; respiratory system |
| carbamezipine | blood; liver; reproductive system |
| carbon tetrachloride (CCl4) | cardiovascular system; kidney; liver; nervous system; |
| | reproductive system |
| ciprofibrate (cipro) | liver |
| clofibrate | muscle |
| cobalt chloride | liver |
| corvastatin | cardiovascular system; liver |
| cyclosporin A | cardiovascular system; kidney; liver; reproductive |
| | system |
| diethylntrosamine | liver |
| dimethylformamide | liver |
| dimethylhydrazine (DMH) | liver |
| diquat | liver |
| ethosuximide | blood |
| etoposide | liver |
| famotidine | liver |
| fluconazole | liver |
| gamfibrozil | muscle |
| ganciclovir | blood; liver |
| hexachloro-1,3-butediene (HCBD) | kidney |
| HIV protease inhibitors | liver |
| hydrazine | liver |
| indomethacin | liver |
| interleukin-6 (IL-6) | liver |
| ketoconazole | liver |
| lead acetate (PbAc) | liver, kidney |
| lipopolysaccharide (LPS) | liver |
| mercury(II) chloride (HgCl₂) | kidney |
| methanol | ocular & visual system |
| methapyrilene | liver |
| methotrexate | liver |
| metronidazole | nervous system; reproductive system |
| miconazole | blood |
| monocrotaline | liver |
| nitric oxide | respiratory system; cardiovascular |
| ondansetron | liver |
| pentamidine | kidney |
| phenobarbital | blood |
| phenylhydrazine (phenylhyrzn) | liver |
| phenytoin | cardiovascular system; nervous system |
| pravastatin | cardiovascular system; liver |
| propulsid | cardiovascular system |
| puromycin aminonucleoside (PAN) | liver; kidney |
| quinolones | liver; nervous system |
| simvastatin | cardiovascular system; liver |
| sodium fluoride (NaF) | liver |
| statins | muscle |
| thioacetamide | liver; kidney |
| tocainidine | cardiovascular system |
| tricyclic antidepressants | cardiovascular system; nervous system |
| troglitazone | liver |
| tumor necrosis factor α (TNFα) | liver |
| uranyl nitrate | kidney |
| valproic acid | reproductive system |
| vincristine | nervous system; cardiovascular system; reproductive |
| | system |
| Wy-16,463 | liver |
| zidovudine (AZT) | blood; liver |
| α-naphthyl isothiocyanate (ANIT) | liver |
| β-naphthoflavone (BNF) | liver |

TABLE 3
The list of biochemical compounds that can be
used in the Targeted Mass Spectrometry platform.
A single run can detect up to 200 compounds from this list.

| Compound | Class |
|---|---|
| acetylcholine | acetic acid amino alcohol ester |
| D-galactose | Aldohexose |
| D-glucose | Aldohexose |
| alpha-D-galactose 1-phosphate | Aldohexose phosphate |
| alpha-D-glucose 6-phosphate | Aldohexose phosphate |
| beta-D-glucose 6-phosphate | Aldohexose phosphate |
| D-glucose 1-phosphate | Aldohexose phosphate |
| D-glucose 6-phosphate | Aldohexose phosphate |
| 5-phosphoribosyl diphosphate | aldopentose phosphate |
| alpha-D-ribose 1-phosphate | aldopentose phosphate |
| alpha-D-ribose 5-phosphate | aldopentose phosphate |
| D-ribose 1-phosphate | aldopentose phosphate |
| D-ribose 5-phosphate | aldopentose phosphate |
| D-erythrose 4-phosphate | Aldotetrose phosphate |
| D-glyceraldehyde 3-phosphate | aldotriose phosphate |
| 5-hydroxy-L-lysine | alpha-amino acid |
| 5-hydroxy-L-tryptophan | alpha-amino acid |
| glycine | alpha-amino acid |
| homocystine | alpha-amino acid |
| L-alanine | alpha-amino acid |
| L-arginine | alpha-amino acid |
| L-argininosuccinate | alpha-amino acid |
| L-asparagine | alpha-amino acid |
| L-aspartate | alpha-amino acid |
| L-citrulline | alpha-amino acid |
| L-cystathionine | alpha-amino acid |
| L-cysteine | alpha-amino acid |
| L-glutamate | alpha-amino acid |
| L-glutamine | alpha-amino acid |
| L-histidine | alpha-amino acid |
| L-homocysteine | alpha-amino acid |
| L-isoleucine | alpha-amino acid |
| L-leucine | alpha-amino acid |
| L-lysine | alpha-amino acid |
| L-methionine | alpha-amino acid |
| L-ornithine | alpha-amino acid |
| L-phenylalanine | alpha-amino acid |
| L-serine | alpha-amino acid |
| L-threonine | alpha-amino acid |
| L-tryptophan | alpha-amino acid |
| L-tyrosine | alpha-amino acid |
| L-valine | alpha-amino acid |
| serotonin | alpha-amino acid derivative |
| choline | amino alcohol |
| ethanolamine | amino alcohol |
| taurine | amino sulfonic acid |
| cysteamine | amino thiol |
| N-carbamoyl-L-aspartate | carbamoyl dicarboxylic acid |
| carbamoyl phosphate | carbamoyl phosphate |
| (R)-3-Hydroxybutanoate | carboxylic acid |
| 2-hydroxybutyrate | carboxylic acid |
| 2-oxoglutarate | carboxylic acid |
| D-lactate | carboxylic acid |
| lactate | carboxylic acid |
| L-lactate | carboxylic acid |
| pantothenate | carboxylic acid |
| pyruvate | carboxylic acid |
| 1,3-bisphospho-D-glycerate | carboxylic acid bisphosphate |
| 2,3-bisphospho-D-glycerate | carboxylic acid bisphosphate |
| (S)-3-Hydroxy-3-methylglutaryl-CoA | carboxylic acid CoA |
| 2-phospho-D-glycerate | carboxylic acid phosphate |
| 3-phospho-D-glycerate | carboxylic acid phosphate |
| dopamine | catecholamine |
| L-adrenaline | catecholamine |
| L-noradrenaline | catecholamine |
| cholate | cholate |
| glycochenodeoxycholate | cholate |
| glycocholate | cholate |
| taurochenodeoxycholate | cholate |
| taurocholate | cholate |
| inositol | cyclic alcohol |
| L-proline | cyclic alpha-imino acid |
| trans-4-hydroxy-L-proline | cyclic alpha-imino acid |
| creatinine | cyclic anhydride |
| chorismate | cyclohexadiene carboxylic acid |
| cytochrome c | cytochrome c |
| 2-deoxy-D-ribose 1-phosphate | deoxyaldopentose phosphate |
| D-malate | dicarboxylic acid |
| fumarate | dicarboxylic acid |
| glutarate | dicarboxylic acid |
| L-malate | dicarboxylic acid |
| oxaloacetate | dicarboxylic acid |
| succinate | dicarboxylic acid |
| GppppG | dinucleotide |
| NAD+ | dinucleotide |
| NADP+ | dinucleotide |
| XppppX | dinucleotide |
| leukotriene A4 | eicosanoid |
| leukotriene B4 | eicosanoid |
| leukotriene C4 | eicosanoid |
| leukotriene D4 | eicosanoid |
| leukotriene E4 | eicosanoid |
| leukotriene F4 | eicosanoid |
| prostaglandin D2 | eicosanoid |
| prostaglandin E2 | eicosanoid |
| prostaglandin F2alpha | eicosanoid |
| prostaglandin G2 | eicosanoid |
| prostaglandin H2 | eicosanoid |
| prostaglandin I2 | eicosanoid |
| thromboxane A2 | eicosanoid |
| thromboxane B2 | eicosanoid |
| arachidonate | fatty acid |
| 4-aminobutanoate | gamma amino acid |
| D-glucono-1,5-lactone 6-phosphate | gluconolactone phosphate |
| 4-hydroxybenzoate | hydroxyphenyl carboxylic acid |
| 4-hydroxyphenylpyruvate | hydroxyphenyl carboxylic acid |
| homogentisate | hydroxyphenyl carboxylic acid |
| histamine | imidazolyl ethylamine |
| sedoheptulose 7-phosphate | ketoheptose phosphate |
| fructose | ketohexose |
| beta-D-fructose 1,6-bisphosphate | ketohexose bisphosphate |
| D-fructose 1,6-bisphosphate | ketohexose bisphosphate |
| D-fructose 2,6-bisphosphate | ketohexose bisphosphate |
| beta-D-fructose 6-phosphate | ketohexose phosphate |
| D-fructose 1-phosphate | ketohexose phosphate |
| D-fructose 6-phosphate | ketohexose phosphate |
| acetone | ketone |
| acetoacetate | ketone carboxylic acid |
| acetoacetyl CoA | ketone CoA |
| D-ribulose 5-phosphate | ketopentose phosphate |
| D-xylulose 5-phosphate | ketopentose phosphate |
| L-ribulose 5-phosphate | ketopentose phosphate |
| L-xylulose 5-phosphate | ketopentose phosphate |
| dihydroxyacetone phosphate | ketotriose phosphate |
| betaine | N,N,N-trimethyl amino acid |
| N,N-dimethylglycine | N,N-dimethyl amino acid |
| S-adenosyl-L-methionine | nucleosidyl alpha-amino acid |
| UDP-D-galactose | nucleotidyl aldohexose |
| UDP-D-glucose | nucleotidyl aldohexose |
| UDP-D-glucuronate | nucleotidyl pyranose carboxylic |
| | acid |
| ascorbate | organic acid |
| isopentenyl diphosphate | organic diphosphate |
| angiotensin I | peptide |
| angiotensin II | peptide |
| bradykinin | peptide |
| lecithin | phospholipid |
| angiotensinogen | protein |
| insulin | protein |
| Renin (Angiotensinogen 1-14) | protein |
| dihydrofolate | pteroyl carboxylic acid |
| folate | pteroyl carboxylic acid |
| tetrahydrofolate | pteroyl carboxylic acid |
| adenine | purine |
| guanine | purine |
| hypoxanthine | purine |
| urate | purine |
| xanthine | purine |
| deoxyadenosine | purine deoxynucleoside |
| deoxyguanosine | purine deoxynucleoside |
| deoxyinosine | purine deoxynucleoside |
| dADP | purine deoxynucleotide |
| dAMP | purine deoxynucleotide |
| dATP | purine deoxynucleotide |
| dGDP | purine deoxynucleotide |
| dGMP | purine deoxynucleotide |
| dGTP | purine deoxynucleotide |
| adenosine | purine nucleoside |
| guanosine | purine nucleoside |
| inosine | purine nucleoside |
| xanthosine | purine nucleoside |
| ADP | purine nucleotide |
| AMP | purine nucleotide |
| ATP | purine nucleotide |
| cAMP | purine nucleotide |
| GDP | purine nucleotide |
| GMP | purine nucleotide |
| GTP | purine nucleotide |
| IMP | purine nucleotide |
| XMP | purine nucleotide |
| XTP | purine nucleotide |
| D-glucuronate | pyranose carboxylic acid |
| nicotinate | pyridine carboxylic acid |
| pyridoxine | pyridine derivative |
| cytosine | pyrimidine |
| dihydrothymine | pyrimidine |
| dihydrouracil | pyrimidine |
| thymine | pyrimidine |
| uracil | pyrimidine |
| L-dihydroorotate | pyrimidine carboxylic acid |
| orotate | pyrimidine carboxylic acid |
| deoxycytidine | pyrimidine deoxynucleoside |
| deoxyuridine | pyrimidine deoxynucleoside |
| thymidine | pyrimidine deoxynucleoside |
| dCDP | pyrimidine deoxynucleotide |
| dCMP | pyrimidine deoxynucleotide |
| dCTP | pyrimidine deoxynucleotide |
| dTDP | pyrimidine deoxynucleotide |
| dTMP | pyrimidine deoxynucleotide |
| dTTP | pyrimidine deoxynucleotide |
| dUDP | pyrimidine deoxynucleotide |
| dUMP | pyrimidine deoxynucleotide |
| dUTP | pyrimidine deoxynucleotide |
| cytidine | pyrimidine nucleoside |
| uridine | pyrimidine nucleoside |
| CDP | pyrimidine nucleotide |
| CMP | pyrimidine nucleotide |
| CTP | pyrimidine nucleotide |
| orotidine 5′-phosphate | pyrimidine nucleotide |
| UDP | pyrimidine nucleotide |
| UMP | pyrimidine nucleotide |
| UTP | pyrimidine nucleotide |
| thiamine diphosphate | pyrimidine thiazole diphosphate |
| sorbitol | sorbitol |
| cholesterol | sterol |
| lanosterol | sterol |
| citrate | tricarboxylic acid |
| isocitrate | tricarboxylic acid |
| glycerol | trihydric alcohol |
| glycerol-3-phosphate | trihydric alcohol phosphate |
| carnitine | trimethylamino carboxylic acid |
| (6R)-5,10-methylenetetrahydrofolate | unassigned |
| 10-formyltetrahydrofolate | unassigned |
| 2-aminoadipate | unassigned |
| 4-Hydroxy-5-Polyprenylbenzoic Acid | unassigned |
| 4-maleylacetoacetate | unassigned |
| 5,10-methylenetetrahydrofolate | unassigned |
| 5-formyltetrahydrofolate | unassigned |
| 5-HPETE | unassigned |
| 5-methyltetrahydrofolate | unassigned |
| 6-phospho-D-gluconate | unassigned |
| 7b-Hydroxycholesterol | unassigned |
| Acetate | unassigned |
| Acetylacetone | unassigned |
| biotin | unassigned |
| Creatine | unassigned |
| farnesyl diphosphate | unassigned |
| Formate | unassigned |
| geranyl diphosphate | unassigned |
| glutathione | unassigned |
| Glycerophosphocholine | unassigned |
| Guanidinoacetic acid | unassigned |
| Hippurate | unassigned |
| Lysophosphatidic acid (LPA) | Unassigned |
| Mevalonic Acid Lactone | Unassigned |
| N-alpha-acetylcitrulline | Unassigned |
| n-valerate | Unassigned |
| oxidized glutathione | Unassigned |
| phosphocholine | Unassigned |
| phosphoenolpyruvate | Unassigned |
| squalene | Unassigned |
| trimethylamine-N-oxide | Unassigned |
| Ubiquinone (CoQ10) | Unassigned |
| 3-ureidopropionate | ureidocarboxylic acid |
| (R)(−)-allantoin | ureidoimidazolidinedione |
| (S)(+)-allantoin | ureidoimidazolidinedione |
| aquacob(III)alamin | vitamin |
| cob(I)alamin | vitamin |
| cob(II)alamin | vitamin |
| cobamide | vitamin |
| hydroxocobalamin | vitamin |

What is claimed is:

1. A method of generating a small molecule efficacy or toxicity profile for an agent, toxicant, or drug, wherein the profile is generated from a subject treated with said agent, toxicant, or drug, said method comprising the steps of:
a. isolating from said treated subject, a biological sample selected from the group consisting of organ, tissue, cell, cellular compartment, organelle, cerebrospinal fluid, synovial fluid, blood and urine;
b. extracting small molecules from said biological sample; and
c. analyzing said small molecules from said sample by mass spectroscopy,
wherein analyzing said small molecules results in the generation of a small molecule efficacy or toxicity profile.

2. The method of claim 1, wherein at least 170 specific small molecules are analyzed by mass spectroscopy.

3. The method of claim 2, wherein said mass spectroscopy analysis is capable of being performed in less than 10 minutes.

4. The method of claim 1, wherein mass spectroscopy analysis comprises staggering injections using a multiple column switching valve, wherein said valve allows combination of different column types into one injection.

5. The method of claim 1, wherein the concentration of each small molecule analyzed is below the concentration of 10 ng/ml.

6. The method of claim 1, which further comprises a computer system for tracking samples for small molecule profiling.

7. The method of claim 1, wherein said treated subject is human.

8. The method of claim 1, wherein said treated subject is a healthy reference subject.

9. The method of claim 1, wherein said treated subject suffers from a disease or disorder.

10. The method of claim 9, wherein said disease or disorder is selected from the group consisting of non-insulin-dependent diabetes (NIDDM), rheumatoid arthritis or inflammation (RA/I), immunological disorder, metabolic disorder, cardiovascular disorder, neurological disorder, oncological disorder, and viral disorder.

11. The method of claim 1, wherein said agent or drug is selected from the group consisting of Chlorpropamide, Tolbuamide, Tolazamide, Acetohexamide, Glyburide, Glipizide, Glimepiride, Pioglitazone, Rosiglitazone, Metformin, Acarbose (Precose), Miglitol (Glycet), Repaglinide (Prandin), Aspirin, Acetaminophen, Ibuprofen, Indomethacin, Peroxicam, Tometin, Rofecoxib, Celecoxib, Valdecoxib, Methotrexate, and Dexamethasone.

12. The method of claim 1, wherein said toxicant is selected from the group consisting of 2,2′,4,4′,5,5′-hexachlorobiphenyl (PCB- 153), 2,3,7,8-tetrachlorodibenzo-p-dioxin (TCDD), 2-bromoethylamine (BEA), 3-methylcholanthrene, 4-aminophenol (PAP), acetaminophen, adriamycin, allyl alcohol, amiodarone, amphotericin B, Aroclor 1254, Aroclor 1260, arsenic, aspirin, astemizole, benzene, cadmium, carbamezipine, carbon tetrachloride (CCl4), ciprofibrate (cipro), clofibrate, cobalt chloride, corvastatin, cyclosporin A, diethylntrosamine, dimethylformamide, dimethylhydrazine (DMH), diquat, ethosuximide, etoposide, famotidine, fluconazole, gamfibrozil, ganciclovir, hexachloro-1,3-butediene (HCBD), HIV protease inhibitors, hydrazine, indomethacin, interleukin-6 (IL-6), ketoconazole, lead acetate (PbAc), lipopolysaccharide (LPS), mercury(II) chloride (HgCl₂), methanol, methapyrilene, methotrexate, metronidazole, miconazole, monocrotaline, nitric oxide, ondansetron, pentamidine, phenobarbital, phenylhydrazine (phenylhyrzn), phenytoin, pravastatin, propulsid, puromycin aminonucleoside (PAN), quinolones, simvastatin, sodium fluoride (NaF), statins, thioacetamide, tocainidine, tricyclic antidepressants, troglitazone, tumor necrosis factor a (TNFα), uranyl nitrate, valproic acid, vincristine, Wy-16,463, zidovudine (AZT), α-naphthyl isothiocyanate (ANIT), and β-naphthoflavone (BNF).

13. A method of generating a small molecule efficacy or toxicity signature for an agent, toxicant, or drug, said method comprising the steps of:
a. obtaining one or more small molecule efficacy or toxicity profiles from one or more treated subjects according to the method of claim 1;
b. obtaining one or more small molecule efficacy or toxicity profiles from one or more control subjects not treated with the agent, toxicant, or drug; and
c. comparing the one or more small molecule efficacy or toxicity profiles from one or more treated subjects to the one or more small molecule efficacy or toxicity profiles generated from the one or more untreated subjects,
wherein comparing said small molecule efficacy or toxicity profiles from the treated and untreated subjects results in the generation of a small molecule efficacy or toxicity signature for the agent, toxicant, or drug.

14. The method of claim 13, wherein said comparing comprises the steps of:
a. generating a data matrix, said matrix comprising two or more analyte/sample values indicating small molecule abundance in said sample;
b. log transforming said data matrix;
c. normalizing said transforming data matrix, said normalization comprising subtracting the median of all analyte/sample values from each analyte/sample value; and
d. performing variance analysis on said normalized data matrix,
thereby generating a small molecule efficacy or toxicity signature.

15. The method of claim 13, wherein said treated subject is human.

16. The method of claim 13, wherein said treated subject is a healthy subject.

17. The method of claim 13, wherein said treated subject suffers from a disease or disorder.

18. The method of claim 17, wherein said disease or disorder is selected from the group consisting of non-insulin-dependent diabetes (NIDDM), rheumatoid arthritis or inflammation (RA/I), immunological disorder, metabolic disorder, cardiovascular disorder, neurological disorder, oncological disorder, and viral disorder.

19. The method of claim 13, wherein said agent or drug is selected from the group consisting of Chlorpropamide, Tolbuamide, Tolazamide, Acetohexamide, Glyburide, Glipizide, Glimepiride, Pioglitazone, Rosiglitazone, Metformin, Acarbose (Precose), Miglitol (Glycet), Repaglinide (Prandin), Aspirin, Acetaminophen, Ibuprofen, Indomethacin, Peroxicam, Tometin, Rofecoxib, Celecoxib, Valdecoxib, Methotrexate, and Dexamethasone.

20. The method of claim 13, wherein said toxicant is selected from the group consisting of 2,2′,4,4′,5,5′-hexachlorobiphenyl (PCB-153), 2,3,7,8-tetrachlorodibenzo-p-dioxin (TCDD), 2-bromoethylamine (BEA), 3-methylcholanthrene, 4-aminophenol (PAP), acetaminophen, adriamycin, allyl alcohol, amiodarone, amphotericin B, Aroclor 1254, Aroclor 1260, arsenic, aspirin, astemizole, benzene, cadmium, carbamezipine, carbon tetrachloride (CCl4), ciprofibrate (cipro), clofibrate, cobalt chloride, corvastatin, cyclosporin A, diethylntrosamine, dimethylformamide, dimethylhydrazine (DMH), diquat, ethosuximide, etoposide, famotidine, fluconazole, gamfibrozil, ganciclovir, hexachloro-1,3-butediene (HCBD), HIV protease inhibitors, hydrazine, indomethacin, interleukin-6 (IL-6), ketoconazole, lead acetate (PbAc), lipopolysaccharide (LPS), mercury(II) chloride (HgCl₂), methanol, methapyrilene, methotrexate, metronidazole, miconazole, monocrotaline, nitric oxide, ondansetron, pentamidine, phenobarbital, phenylhydrazine (phenylhyrzn), phenytoin, pravastatin, propulsid, puromycin aminonucleoside (PAN), quinolones, simvastatin, sodium fluoride (NaF), statins, thioacetamide, tocainidine, tricyclic antidepressants, troglitazone, tumor necrosis factor a (TNFα), uranyl nitrate, valproic acid, vincristine, Wy-16,463, zidovudine (AZT), α-naphthyl isothiocyanate (ANIT), and β-naphthoflavone (BNF).

21. A method of generating a small molecule disease profile from a subject suffering from a known or unknown disease or disorder, said method comprising the steps of:
a. isolating from said diseased subject, a biological sample selected from the group consisting of organ, tissue, cell, cellular compartment, organelle, cerebrospinal fluid, synovial fluid, blood and urine;
b. extracting small molecules from said biological sample; and
c. analyzing said small molecules from said sample by mass spectroscopy,
wherein analyzing said small molecules results in the generation of a small molecule disease profile.

22. The method of claim 21, wherein at least 170 specific small molecules are analyzed by mass spectroscopy.

23. The method of claim 22, wherein said mass spectroscopy analysis is capable of being performed in less than 10 minutes.

24. The method of claim 21, wherein mass spectroscopy analysis comprises staggering injections using a multiple column switching valve, wherein said valve allows combination of different column types into one injection.

25. The method of claim 21, wherein the concentration of each small molecule is below the concentration of 10 ng/ml.

26. The method of claim 21, which further comprises a computer system for tracking samples for small molecule profiling.

27. The method of claim 21, wherein said diseased subject is human.

28. The method of claim 27, wherein said disease or disorder is selected from the group consisting of non-insulin-dependent diabetes (NIDDM), rheumatoid arthritis or inflammation (RA/I), immunological disorder, metabolic disorder, cardiovascular disorder, neurological disorder, oncological disorder, and viral disorder.

29. A method of generating a small molecule disease signature for a disease or disorder, said method comprising the steps of:
a. obtaining one or more small molecule disease profiles for a subject suffering from a disease or disorder according to the method of claim 21;
b. obtaining one or more small molecule profiles from one or more non-diseased control subjects, wherein the control subjects do not suffer from the disease or disorder; and
c. comparing the one or more small molecule disease profiles from one or more diseased subjects to the one or more small molecule disease profiles generated from one or more non-diseased control subjects,
wherein comparing said small molecule disease profiles from the diseased subjects and non-diseased control subjects results in the generation of a small molecule disease signature for the disease or disorder.

30. The method of claim 29, wherein said comparing comprises the steps of:
a. generating a data matrix, said matrix comprising two or more analyte/sample values indicating small molecule abundance in said sample;
b. log transforming said data matrix;
c. normalizing said transforming data matrix, said normalization comprising subtracting the median of all analyte/sample values from each analyte/sample value; and
d. performing variance analysis on said normalized data matrix,
thereby generating a small molecule disease signature.

31. The method of claim 29, wherein said subject is human.

32. The method of claim 29, wherein said disease or disorder is selected from the group consisting of non-insulin-dependent diabetes (NIDDM), rheumatoid arthritis or inflammation (RA/I), immunological disorder, metabolic disorder, cardiovascular disorder, neurological disorder, oncological disorder, and viral disorder.

33. A method of predicting the efficacy of an agent or drug with an unknown efficacy, said method comprising the steps of:
a. generating a first small molecule efficacy signature of the agent or drug according to the method of claim 13;
b. obtaining one or more second small molecule efficacy signatures, wherein the one or more second small molecule efficacy signatures have been generated with agents or drugs with known efficacies;
comparing said first small molecule efficacy signature to the one or more second small molecule efficacy signatures, thereby predicting the efficacy of said agent or drug.

34. The method of claim 33, wherein the one or more second small molecule efficacy signatures are a database of small molecule efficacy signatures.

35. The method of claim 33, wherein the first and second small molecule efficacy signatures are similar, thereby predicting that the agent or drug with an unknown efficacy will have an efficacy similar to the agents or drugs with known efficacies used to generate the second small molecule efficacy profiles.

36. The method of claim 33, wherein the first and second small molecule efficacy signatures are different, thereby predicting that the agent or drug with an unknown efficacy will not have an efficacy similar to the agents or drugs with known efficacies used to generate the second small molecule efficacy profiles.

37. A method of determining the toxicity of an agent or drug with an unknown toxicity, said method comprising the steps of:
a. generating a first small molecule toxicity signature of the agent or drug according to the method of claim 13;
b. obtaining one or more second small molecule toxicity signatures, wherein the one or more second small molecule toxicity signatures have been generated with agents or drugs with known toxicities; and
c. comparing said first small molecule toxicity signature to the one or more second small molecule toxicity signatures,
thereby determining the toxicity of said agent or drug.

38. The method of claim 37, wherein the one or more second small molecule toxicity signatures are a database of small molecule toxicity signatures.

39. The method of claim 37, wherein the first and second small molecule toxicity signatures are similar, thereby determining that the agent or drug with an unknown toxicity will have a toxicity similar to the agents or drugs with known toxicities used to generate the second small molecule toxicity signatures.

40. The method of claim 37, wherein the first and second small molecule toxicity signatures are different, thereby predicting that the agent or drug with an unknown toxicity will not have a toxicity similar to the agents or drugs with known toxicities used to generate the second small molecule toxicity signatures.

41. A method of diagnosing a disease or disorder in a subject with an unknown disease or disorder, said method comprising the steps of:
a. generating a small molecule disease profile of the subject according to the method of claim 21;
b. obtaining one or more small molecule disease signatures according to claim 29, wherein the one or more small molecule disease signatures have been generated from subjects with known diseases or disorders; and
c. comparing said small molecule disease profile to the one or more small molecule disease signatures,
thereby diagnosing the disease or disorder.

42. The method of claim 41, wherein the one or more small molecule disease signatures are a database of small molecule disease signatures.

43. The method of claim 41, wherein the small molecule disease profile of the subject and the small molecule disease signatures are similar, thereby diagnosing the subject with the disease or disorder of the subjects used to generate the small molecule disease signatures.

44. The method of claim 41, wherein the small molecule disease profile of the subject and the small molecule disease signatures are different, thereby diagnosing that the subject does not have the disease or disorder of the subjects used to generate the small molecule disease signatures.

45. A method of monitoring the progression or remission of a disease or disorder in a subject undergoing treatment for said disease or disorder, said method comprising the steps of:
a. obtaining a small molecule disease profile from said subject at the onset of treatment for said disease or disorder;
b. obtaining small molecule disease profiles from said subject at multiple times during the course of treatment for said disease or disorder; and
c. comparing the small molecule disease profiles from step b) with the small molecule disease profile obtained at the onset of treatment,
thereby measuring the effectiveness of said treatment and monitoring the progression or remission of said disease or disorder in said subject.
